(19) United States
(12) Patent Application Publication
(10) Pub. No.: US 2019/0022218 A1
(43) Pub. Date: Jan. 24, 2019

(54) BIOPHOTONIC COMPOSITIONS FOR THE TREATMENT OF PYODERMA

(71) Applicant: KLOX TECHNOLOGIES LIMITED, Dublin (IE)

(72) Inventors: Francesco BELLINI, Calgary (CA); Andrea SPATERNA, Matelica (IT); Andrea MARCHEGIANI, Matelica (IT); Nikolaos LOUPIS, Athens (GR); Remigio PIERGALLINI, San Benedetto del Tronto (IT)

(73) Assignee: KLOX TECHNOLOGIES LIMITED, Dublin (IE)

(21) Appl. No.: 16/068,709

(22) PCT Filed: Jan. 11, 2017
(86) PCT No.: PCT/CA2017/050034

§ 371 Date: Jul. 9, 2018

Related U.S. Application Data
(60) Provisional application No. 62/277,272, filed on Jan. 11, 2016.

(51) Int. Cl. A61K 41/00; A61P 31/04; A61K 9/00; A61K 33/40; A61K 31/728; A61K 31/7004; A61K 31/4166; A61K 33/00; A61K 31/327; A61K 47/18; A61N 5/06

(52) U.S. Cl. CPC A61K 41/0057; A61P 31/04; A61K 9/0014; A61K 33/40; A61K 31/728; A61K 31/7004; A61N 2005/067; A61K 33/00; A61K 31/327; A61K 47/183; A61N 5/062; A61N 2005/0662; A61K 31/4166

(57) ABSTRACT

The present document describes methods and uses of biophotonic compositions which comprise at least one oxidant and at least one chromophore capable of activating the oxidant, in association with a pharmacologically acceptable carrier for the treatment of pyoderma, deep pyoderma, or antibiotic resistant pyoderma.

### RELATED APPLICATIONS

This application claims priority to and benefit from U.S. Provisional Patent Application No. 62/277,272, filed Jan. 11, 2016, the disclosure of which is incorporated herein by reference in its entirety.

### TECHNICAL FIELD

This description relates to the field of biophotonic compositions, methods, and uses for treating pyoderma.

### BACKGROUND

Cutaneous bacterial infections (pyoderma) are very frequent in dogs and cats because the skin of these animals has a very thin stratum corneum and a follicular ostium poorly protected by the hydrolipidic film. This results in scarcely effective physicochemical protection, especially in the presence of predisposing diseases (allergies, endocrinopathies, etc.), and the skin is not able to prevent bacterial multiplication and skin invasion. The bacteria involved in the infection are mainly Staphylococcus pseudointermedius but also S. aureus, S. hyicus, S. schleileri and less frequently streptococci, Proteus spp., Pseudomonas spp., Escherichia coli. There is a higher risk of developing a pyoderma infection in animals that have a pre-existing fungal infection or an endocrine disease such as hyperthyroidism, or that have an allergy to a food ingredient or ingredients, or a parasitic infection such as by a Demodex spp.

Several types of pyoderma exist:

Surface pyoderma, which is excessive bacterial proliferation confined to the skin surface (skin fold pyoderma and acute moist dermatitis);

Superficial pyoderma, where the bacterial infection is present in the hair follicles, without invasion of the dermis (bacterial folliculitis, mucocutaneous pyoderma and impetigo); and

Deep pyoderma, in which the infectious process has gone beyond the basal membrane, and deeply involves the dermis, with the formation of piogranulomatous (boils) or diffuse (cellulite) lesions, that both tend to fistulize. The location of the lesions determines their classification. Classifications include: nasal furunculosis, chin furunculosis, interdigital or podal furunculosis, pyotraumatic furunculosis, furunculosis and cellulitis localized or generalized.

While surface and superficial pyoderma do not represent a serious problem for the veterinary dermatologist, as they are generally responsive to antibiotic therapy (topical and/or systemic), deep pyoderma is still a difficult problem that necessitates systemic antibiotic treatment lasting several weeks/months. Furthermore, another serious problem is antibiotic-resistant pyoderma (so-called methicillin-resistant bacteria).

Effective treatments of pyoderma, deep pyoderma, and antibiotic resistant pyoderma are needed.

### SUMMARY OF THE DISCLOSURE

In some aspects, the disclosure provides a method of treating pyoderma, deep pyoderma, or antibiotic-resistant pyoderma comprising: applying a biophotonic composition to a patient in need thereof, wherein the biophotonic composition comprises at least one oxidant and at least one chromophore capable of activating the oxidant; and exposing said biophotonic composition to actinic light for a time sufficient for said chromophore to cause activation of said oxidant. In certain such aspects, the patient is a mammal, such as a feline or a canine. In certain such aspects, the composition is applied to the patient's skin, such as one or more times per week for one or more weeks. In certain such aspects, the method is performed once per week for one or more weeks, such as one week, two weeks, three weeks, four weeks, five weeks, or six weeks. In certain such aspects, the method is performed twice per week for one or more weeks, such as one week, two weeks, three weeks, four weeks, five weeks, or six weeks.

In some embodiments, said biophotonic composition is exposed to actinic light for a period of less than about 5 minutes, e.g., for a period of from about 1 second to about 5 minutes. In certain such embodiments, said biophotonic composition is exposed to actinic light for a period of less than about 5 minutes per cm² of an area to be treated, e.g., for a period of about 1 second to about 5 minutes per cm².

In some embodiments, the source of actinic light is positioned over an area to be treated. In some embodiments, said actinic light is visible light having a wavelength between about 400 nm and about 700 nm.

In some embodiments, the oxidant present in the biophotonic composition is chosen from hydrogen peroxide, carbamide peroxide and benzoyl peroxide. In other embodiments, the oxidant is chosen from a peroxy acid and an alkali metal percarbonate.

In some embodiments, the biophotonic composition further comprises at least one healing factor chosen from hyaluronic acid, glucosamine, and allantoin.

In some embodiments, the biophotonic composition further comprises at least one gelling agent, such as glucose, modified starch, methyl cellulose, carboxymethyl cellulose, propyl cellulose, hydroxypropyl cellulose, a carbomer, alginic acid, sodium alginate, potassium alginate, ammonium alginate, calcium alginate, agar, carrageenan, locust bean gum, pectin, or gelatin.

In some embodiments, the chromophore of the biophotonic composition is chosen from a xanthene derivative dye, an azo dye, a biological stain, and a carotenoid. In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B, i.e., Eosin Y), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from saffranin O, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthin, lycopene, α-carotene, β-carotene, bixin, and fucoxanthine. In certain such embodiments, said carotenoid is present in the composition as a mixture chosen from saffron red powder, annatto extract, and brown algae extract.

In certain embodiments, said biophotonic composition further comprises at least one chelating agent chosen from ethylenediaminetetraacetic acid (EDTA) and ethylene glycol tetraacetic acid (EGTA).

In some aspects, the disclosure provides for use of a biophotonic composition for the manufacture of a medicament for treating a patient afflicted with pyoderma, deep pyoderma, or antibiotic resistant pyoderma, wherein said composition comprises: at least one oxidant, and at least one chromophore capable of activating the oxidant; in association with a pharmacologically acceptable carrier. In certain such aspects, the patient is a mammal, such as a feline or a canine.

In some aspects, the disclosure provides for use of a biophotonic composition for the treatment of a patient afflicted with pyoderma, deep pyoderma, or antibiotic resistant pyoderma, wherein said composition comprises: at least one oxidant; and at least one chromophore capable of activating the oxidant in association with a pharmacologically acceptable carrier. In certain such aspects, the patient is a mammal, such as a feline, or a canine.

### Definitions

Before continuing to describe the present disclosure in further detail, it is to be understood that this disclosure is not limited to specific compositions or process steps, as such may vary. It must be noted that, as used in this specification and the appended embodiments, the singular form “a”, “an” and “the” include plural referents unless the context clearly dictates otherwise.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Biophotonic” means the generation, manipulation, detection and application of photons in a biologically relevant context. In other words, biophotonic compositions exert their physiological effects primarily due to the generation and manipulation of photons. “Biophotonic composition” is a composition as described herein that may be activated by light to produce photons for biologically relevant applications.

“Topical” means as applied to body surfaces, such as the skin, mucous membranes, vagina, oral cavity, internal surgical wound sites, and the like.

Terms “chromophore,” “photoactivating agent” and “photoactivator” are used herein interchangeably. A chromophore means a compound, when contacted by light irradiation, is capable of absorbing the light. The chromophore readily undergoes photoexcitation and can then transfer its energy to other molecules or emit it as light.

The term “oxidant” is intended to mean either a compound that readily transfers oxygen atoms to and thus, oxidizes other compounds, or a substance that gains electrons in a redox chemical reaction.

The term “chelating agent” is intended to mean a compound that binds metal ions, such as iron, cobalt, copper, manganese, and chromium, and facilitates their solvation in solution.

The term “healing factor” is intended to mean a compound that promotes or enhances the healing or regenerative process of a tissue.

The term “active oxygen species” is intended to mean chemically-reactive molecules containing oxygen. Examples include, but are not limited to, oxygen ions and peroxides. They can be either inorganic or organic. Active oxygen species are highly reactive due to the presence of unpaired valence shell electrons. They are also referred to as “reactive oxygen,” “active oxygen,” or “reactive oxygen species.”

Features and advantages of the subject matter hereof will become more apparent in light of the following detailed description of selected embodiments, as illustrated in the accompanying figures. As will be realized, the subject matter disclosed is capable of modifications in various respects, all without departing from the scope of the disclosed embodiments. Accordingly, the drawings and the description are to be regarded as illustrative in nature and not as restrictive and the full scope of the subject matter is set forth in the disclosed embodiments.

### BRIEF DESCRIPTION OF THE DRAWINGS

Further features and advantages of the present disclosure will become apparent from the following detailed description, taken in combination with the appended drawings, in which:

FIG. 1 illustrates the Stokes' shift.

FIG. 2 illustrates the absorption and emission spectra of donor and acceptor chromophores. The spectral overlap between the absorption spectrum of the acceptor chromophore and the emission spectrum of the donor chromophore is also shown.

FIG. 3 is a schematic of a Jablonski diagram that illustrates the coupled transitions involved between a donor emission and acceptor absorbance.

FIGS. 4A-4D show photographs of the treatment area over time in case 1, a canine patient. FIG. 4A: left forelimb foot treated with a composition of the present disclosure comprising 12% urea peroxide (UP), with actinic light illumination for 2 minutes at a 5 cm distance; FIG. 4B: left hindlimb foot treated with a composition of the present description comprising 3% UP, with actinic light illumination for 2 minutes at a 5 cm distance; FIG. 4C: right forelimb foot treated with a composition of the present description comprising 6% UP, with actinic light illumination for 2 minutes at a 5 cm distance FIG. 4D: right hindlimb untreated as control.

FIGS. 5A-5G show photographs of the treatment area over time in case 2, a canine patient. FIG. 5A: dorsal region of the neck (right side) treated with a composition of the present disclosure comprising 6% UP, with Thera™ lamp illumination for 2 minutes at a 5 cm distance; FIG. 5B: dorsal region of the neck (right side) 8 weeks after treatment; FIG. 5C: ventral region of the neck (right side) treated with a composition of the present description comprising 6% UP, with Thera™ lamp illumination for 2 minutes at a 5 cm distance; FIG. 5D: ventral region of the neck (right side) 8 weeks after treatment; FIG. 5E: ventral region of the neck untreated as control; FIG. 5F: right forelimb foot treated with a composition of the present disclosure comprising 6% UP, with Thera™ lamp illumination for 2 minutes at a 5 cm distance; FIG. 5G: right forelimb foot 8 weeks after treatment.

FIGS. 6A-6B show photographs of the treatment areas over time in case 3, a canine patient. FIG. 6A: left hindlimb foot treated with a composition of the present disclosure comprising 6% UP, with Thera™ lamp illumination for 2 minutes at a 5 cm distance; FIG. 6B: right hindlimb foot untreated as control.

FIG. 7 shows photographs of the treatment area over time in case 4, a canine patient. Dorsum area treated with a composition of the present disclosure comprising 6% UP, with Thera™ lamp illumination for 2 minutes at a 5 cm distance. For the second treatment, the area was treated with a composition of the present disclosure comprising 3% UP and illuminated the same.

FIG. 8 shows photographs of the treatment area over time in case 5, a canine patient. Right hindlimb foot treated with a composition of the present description comprising 6% UP, with Thera™ lamp illumination for 2 minutes at a 5 cm distance.

FIGS. 9A-9B show photographs of the treatment area over time in case 6, a canine patient. FIG. 9A: left hindlimb foot treated with a composition of the present disclosure comprising 6% UP, with Thera™ lamp illumination for 2 minutes at a 5 cm distance; FIG. 9B: right hindlimb foot untreated as control.

FIG. 10 shows photographs of the treatment area over time in case 7, a canine patient. Right inguinal area treated with a composition of the present disclosure comprising 3% UP, with Thera lamp illumination for 2 minutes at a 5 cm distance.

FIG. 11 shows photographs of the treatment area over time in case 9, a canine patient. Inguinal area treated with a composition of the present disclosure comprising 6% UP, with Thera™ lamp illumination for 2 minutes at a 5 cm distance (from left to right, before treatment, immediately after treatment, a week after treatment).

FIGS. 12A-12B show photographs of the treatment area over time in case 10, a canine patient. FIG. 12A: left hindlimb foot treated with a composition of the present disclosure comprising 6% UP, with Thera™ lamp illumination for 2 minutes at a 5 cm distance; FIG. 12B: left hindlimb tarsus treated with a composition of the present description comprising 6% UP, with Thera™ lamp illumination for 2 minutes at a 5 cm distance.

FIGS. 13A-13B show photographs of the treatment area over time in case 12, a canine patient. FIG. 13A: left tarsus treated with treated with a composition of the present disclosure comprising 6% UP, with Thera™ lamp illumination for 2 minutes at a 5 cm distance; FIG. 13B: right tarsus treated with treated with a composition of the present disclosure comprising 6% UP, with Thera™ lamp illumination for 2 minutes at a 5 cm distance.

FIG. 14 shows the total bacteria count in the tarsus lesion area in case 10.

FIGS. 15A-15B show the total bacteria count in the tarsus lesion area in case 12. FIG. 15A: counts in left tarsus lesion; FIG. 15B: counts in right tarsus lesion.

### DETAILED DESCRIPTION

In some aspects, the disclosure provides a method of treating pyoderma, deep pyoderma, or antibiotic resistant pyoderma comprising: applying a biophotonic composition to a patient in need thereof, wherein the biophotonic composition comprises at least one oxidant and at least one chromophore capable of activating the oxidant; and exposing said biophotonic composition to actinic light for a time sufficient for said chromophore to cause activation of said oxidant. In certain such aspects, the patient is a mammal, such as a feline or a canine.

In other aspects, the disclosure provides for use of a biophotonic composition for the manufacture of a medicament for treating a patient afflicted with pyoderma, deep pyoderma, or antibiotic resistant pyoderma, wherein said composition comprises: at least one oxidant, and at least one chromophore capable of activating the oxidant; in association with a pharmacologically acceptable carrier. In certain such aspects, the patient is a mammal, such as a feline or a canine.

In some other aspects, the disclosure provides for use of a biophotonic composition for the treatment of a patient afflicted with pyoderma, deep pyoderma, or antibiotic-resistant pyoderma, wherein said composition comprises: at least one oxidant; and at least one chromophore capable of activating the oxidant; in association with a pharmacologically acceptable carrier. In certain such aspects, the patient is a mammal, such as a feline or a canine.

### Biophotonic Compositions

The present disclosure provides methods and uses comprising biophotonic compositions for treating pyoderma, deep pyoderma, or antibiotic resistant pyoderma. Biophotonic compositions are compositions that are, in a broad sense, activated by light (e.g., photons) of specific wavelength. These compositions contain at least one exogenous chromophore which is activated by light and accelerates the dispersion of light energy, which leads to light carrying on a therapeutic effect on its own, and/or to the photochemical activation of other agents contained in the composition. The composition may comprise an agent which, when mixed with a chromophore or combination of chromophores and subsequently activated by light, can be photochemically activated, which may lead to the formation of oxygen radicals, such as singlet oxygen.

In some aspects, the disclosure provides a method of treating pyoderma, deep pyoderma, or antibiotic resistant pyoderma comprising: applying a biophotonic composition to a patient in need thereof, wherein the biophotonic composition comprises at least one oxidant and at least one chromophore capable of activating the oxidant: and exposing said biophotonic composition to actinic light for a time sufficient for said chromophore to cause activation of said oxidant.

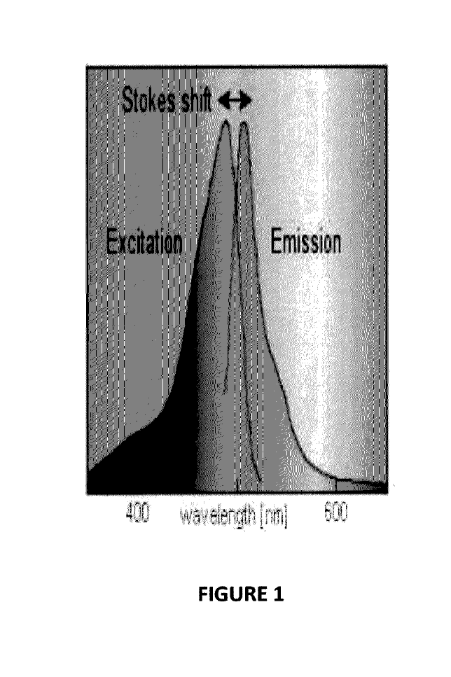

When a chromophore absorbs a photon of a certain wavelength, it becomes excited. This is an unstable condition and the molecule tries to return to the ground state, giving away the excess energy. For some chromophores, it is favorable to emit the excess energy as light when transforming back to the ground state. This process is called fluorescence. The peak wavelength of the emitted fluorescence is shifted towards longer wavelengths compared to the absorption wavelengths (‘Stokes' shift’). The emitted fluorescent energy can then be transferred to the other components of the composition or to a treatment site on to which the biophotonic composition is topically applied. Differing wavelengths of light may have different and complementary therapeutic effects on tissue. Stokes' shift is illustrated in FIG. 1.

Without being bound to theory, it is thought that fluorescent light emitted by photoactivated chromophores may have therapeutic properties due to its femto-, pico- or nano-second emission properties which may be recognized by biological cells and tissues, leading to favorable biomodulation. Furthermore, the emitted fluorescent light has a longer wavelength and hence a deeper penetration into the tissue than the activating light. Irradiating tissue with such a broad range of wavelengths, including in some embodiments the activating light which passes through the composition, may have different and complementary effects on the cells and tissues. Moreover, in some embodiments of the composition containing oxidants, micro-bubbling within the composition has been observed which may be associated with the generation of oxygen species by the photoactivated chromophores. This may have a physical impact on the tissue to which it is applied, for example by dislodging biofilm and debridement of necrotic tissue or providing a pressure stimulation. The biofilm can also be pre-treated with an oxygen-releasing agent to weaken the biofilm before treating with the composition of the present disclosure.

In certain embodiments, the biophotonic compositions of the present disclosure are substantially transparent/translucent and/or have high light transmittance in order to permit light dissipation into and through the composition. In this way, the area of tissue under the composition can be treated both with the fluorescent light emitted by the composition and the light irradiating the composition to activate it, which may benefit from the different therapeutic effects of light having different wavelengths.

The % transmittance of the biophotonic composition can be measured in the range of wavelengths from about 250 nm to about 800 nm using, for example, a Perkin-Elmer Lambda™ 9500 series UV-visible spectrophotometer. Alternatively, a Synergy™ HT spectrophotometer (BioTek Instrument, inc.) can be used in the range of wavelengths from 380 nm to 900 nm.

Transmittance is calculated according to the following equation:

$${A}_{λ} = {log}_{10} \frac{{I}_{0}}{I} = {log}_{10} \frac{1}{T} .$$

where A is absorbance, T is transmittance, I₀ is intensity of radiation before passing through material, and I is intensity of light passing through material.

The values can be normalized for thickness. As stated herein, % transmittance (translucency) is as measured for a 2 mm thick sample at a wavelength of 526 nm. It will be clear that other wavelengths can be used.

Embodiments of the biophotonic compositions of the present disclosure are for topical uses. The biophotonic composition can be in the form of a semi-solid or viscous liquid, such as a gel, or are gel-like, and which have a spreadable consistency at room temperature (e.g., about 20-25° C.), prior to illumination. By spreadable is meant that the composition can be topically applied to a treatment site at a thickness of less than about 0.5 mm, or from about 0.5 mm to about 3 mm, from about 0.5 mm to about 2.5 mm, or from about 1 mm to about 2 mm. In some embodiments, the composition can be topically applied to a treatment site at a thickness of about 2 mm or about 1 mm. Spreadable compositions can conform to the topography of a treatment site. This can have advantages over a non-conforming material in that a better and/or more complete illumination of the treatment site can be achieved and the compositions are easy to apply and remove.

These compositions may be described based on the components making up the composition. Additionally or alternatively, the compositions of the present disclosure have functional and structural properties and these properties may also be used to define and describe the compositions. Individual components of the composition of the present disclosure are detailed as below.

### Oxidants

In some embodiments, the biophotonic compositions of the present disclosure comprise one or more oxidants. The biophotonic compositions of the present disclosure comprise oxidants as a source of oxygen radicals. For instance, peroxide compounds are oxidants that contain the peroxy group (R—O—O—R), which is a chainlike structure containing two oxygen atoms, each of which is bonded to the other and a radical or some element. In some embodiments, the biophotonic compositions of the present disclosure comprises one or more oxidants selected from, but are not limited to, hydrogen peroxide, urea hydrogen peroxide, benzoyl peroxide, peroxy acids and/or alkali metal percarbonates.

Suitable oxidants for the biophotonic compositions of this disclosure include, but are not limited to:

Hydrogen peroxide (H₂O₂) is the starting material to prepare organic peroxides. H₂O₂ is a powerful oxidizing agent, and the unique property of hydrogen peroxide is that it breaks down into water and oxygen and does not form any persistent, toxic residual compound. Hydrogen peroxide for use in this composition can be used in a gel, for example with 6% hydrogen peroxide by weight of the total composition. A suitable range of concentration over which hydrogen peroxide can be used in a composition of the present disclosure is less than about 12% by weight of the total compositions. In some embodiments, hydrogen peroxide is present in an amount from about 0.1% to about 12%, from about 1% to about 12%, from about 3.5% to about 12%, from about 3.5% to about 6% or from about 0.1% to about 6% by weight of the total composition.

Urea hydrogen peroxide (also known as urea peroxide, carbamide peroxide or percarbamide) is soluble in water and contains about 36% hydrogen peroxide. Carbamide peroxide for use in this composition can be used as a gel, for example with about 16% carbamide peroxide that represents about 5.6% hydrogen peroxide. A suitable range of concentration over which urea peroxide can be used in a composition of the present disclosure is less than about 36% by weight of the total composition. In some embodiments, urea peroxide is present in an amount of from about 0.3% to about 36%, from about 3% to about 36%, or from about 10% to about 36%, or from about 3% to about 16% or from about 0.3% to about 16% by weight of the total composition.

In some embodiments, urea peroxide is present in an amount of about 2% by weight of the total composition. In some embodiments, urea peroxide is present in an amount of about 3% by weight of the total composition. In some embodiments, urea peroxide is present in an amount of about 6% by weight of the total composition. In some embodiments, urea peroxide is present in an amount of about 8% by weight of the total composition. In some embodiments, urea peroxide is present in an amount of about 12% by weight of the total composition. Urea peroxide brakes down to urea and hydrogen peroxide in a slow-release fashion that can be accelerated with heat or photochemical reactions. The released urea (carbamide, (NH₂)₂CO), is highly soluble in water and is a powerful protein denaturant. It increases solubility of some proteins and enhances rehydration of the skin and/or mucosa.

Benzoyl peroxide consists of two benzoyl groups (benzoic acid with the H of the carboxylic acid removed) joined by a peroxide group. The released peroxide groups are effective at killing bacteria. Benzoyl peroxide also promotes skin turnover and clearing of pores. Benzoyl peroxide breaks down to benzoic acid and oxygen upon contact with skin, neither of which is toxic. A suitable range of concentration over which benzoyl peroxide can be used in the present composition is less than about 10% by weight of the total composition, such as from about 1% to about 10%, from about 1% to about 8%, from about 2.5% to about 5%. In some embodiments, benzoyl peroxide is present in an amount from about 1% to about 10%, from about 1% to about 8%, or from about 2.5% to about 5% by weight of the total composition.

In some embodiments, suitable oxidants may also include peroxy acids and alkali metal percarbonates, but the inclusion of any other forms of peroxides (e.g., organic or inorganic peroxides) should be avoided due to their increased toxicity and their unpredictable reaction with the photodynamic energy transfer.

### Chromophores/Photoactivators

In some embodiments, the biophotonic compositions of the present disclosure comprise one or more chromophores, which can be considered exogenous, e.g., are not naturally present in skin or tissue. When a biophotonic composition of the present disclosure is illuminated with light, the chromophore(s) are excited to a higher energy state. When the chromophore(s)' electrons return to a lower energy state, they emit photons with a lower energy level, thus causing the emission of light of a longer wavelength (Stokes' shift). In the proper environment, some of this energy release is transferred to oxygen and causes the formation of oxygen radicals, such as singlet oxygen.

Suitable chromophores for the biophotonic compositions of the disclosure can be fluorescent dyes (or stains), although other dye groups or dyes (biological and histological dyes, food colorings, carotenoids, naturally occurring fluorescent and other dyes) can also be used.

In some embodiments, the biophotonic composition of the present disclosure comprises a chromophore which undergoes partial or complete photobleaching upon application of light. By photobleaching is meant a photochemical destruction of the chromophore which can generally be characterized as a visual loss of color or loss of fluorescence.

In some embodiments, the chromophore absorbs at a wavelength in the range of the visible spectrum, such as at a wavelength of from about 380 to about 800 nm, from about 380 to about 700 nm, or from about 380 to about 600 nm. In some embodiments, the chromophore absorbs at a wavelength of from about 200 to about 800 nm, from about 200 to about 700 nm, from about 200 to about 600 nm, or from about 200 to about 500 nm. In some embodiments, the chromophore absorbs at a wavelength of from about 200 to about 600 nm. In some embodiments, the chromophore absorbs light at a wavelength of from about 200 to about 300 nm, from about 250 to about 350 nm, from about 300 to about 400 nm, from about 350 to about 450 nm, from about 400 to about 500 nm, from about 400 to about 600 nm, from about 450 to about 650 nm, from about 600 to about 700 nm, from about 650 to about 750 nm or from about 700 to about 800 nm.

In some embodiments, the chromophore or combination of chromophores is present in an amount of from about 0.001% to about 40% by weight of the total composition. In some embodiments, the chromophore or combination of chromophores is present in an amount of from about 0.005% to about 2%, from about 0.01% to about 1%, from about 0.01% to about 2%, from about 0.05% to about 1%, from about 0.05% to about 2%, from about 0.1% to about 1%, from about 0.1% to about 2%, from about 1% to about 5%, from about 2.5% to about 7.5%, from about 5% to about 10%, from about 7.5% to about 12.5%, from about 10% to about 15%, from about 12.5% to about 17.5%, from about 15% to about 20%, from about 17.5% to about 22.5%, from about 20 to about 25%, from about 22.5% to about 27.5%, from about 25% to about 30%, from about 27.5% to about 32.5%, from about 30% to about 35%, from about 32.5% to about 37.5%, or from about 35% to about 40% by weight of the total composition. In some embodiments, the chromophore or combination of chromophores is present in an amount of at least about 0.2% by weight of the total composition.

In some embodiments, the chromophore or combination of chromophores is present in an amount of from 0.001 to 40% by weight of the total composition. In some embodiments, the chromophore or combination of chromophores is present in an amount of from 0.005% to 2%, from 0.01% to 1%, from 0.01% to 2%, from 0.05% to 1%, from 0.05% to 2%, from 0.1% to 1%, from 0.1% to 2%, from 1% to 5%, from 2.5% to 7.5%, from 5% to 10%, from 7.5% to about 12.5%, from 10% to about 15%, from 12.5% to about 17.5%, from 15% to about 20%, from 17.5% to about 22.5%, from 20% to about 25%, from 22.5% to about 27.5%, from 25% to about 30%, from 27.5% to about 32.5%, from 30% to about 35%, from 32.5% to about 37.5%, or from 35% to about 40% by weight of the total composition. In some embodiments, the chromophore or combination of chromophores is present in an amount of at least 0.2% by weight of the total composition.

It will be appreciated to those skilled in the art that optical properties of a particular chromophore may vary depending on the chromophore's surrounding medium. Therefore, as used herein, a particular chromophore's absorption and/or emission wavelength (or spectrum) corresponds to the wavelengths (or spectra) measured in a biophotonic composition of the present disclosure.

In some embodiments, the biophotonic compositions disclosed herein may include at least one additional chromophore. Combining chromophores may increase photo-absorption by the combined dye molecules and enhance absorption and photo-biomodulation selectivity. This creates multiple possibilities of generating new photosensitive, and/or selective chromophores mixtures.

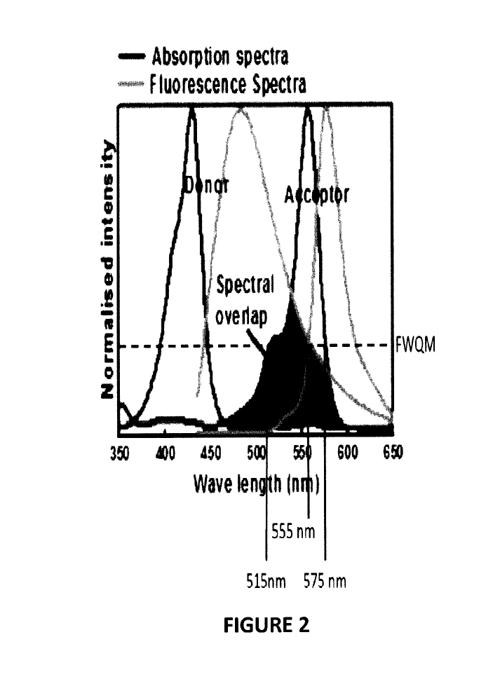

When such multi-chromophore compositions are illuminated with light, energy transfer can occur between the chromophores. This process, known as resonance energy transfer, is a photophysical process through which an excited ‘donor’ chromophore (also referred to herein as first chromophore) transfers its excitation energy to an ‘acceptor’ chromophore (also referred to herein as second chromophore). The efficiency and directedness of resonance energy transfer depends on the spectral features of donor and acceptor chromophores. In particular, the flow of energy between chromophores is dependent on a spectral overlap reflecting the relative positioning and shapes of the absorption and emission spectra. For energy transfer to occur the emission spectrum of the donor chromophore overlap with the absorption spectrum of the acceptor chromophore (FIG. 2).

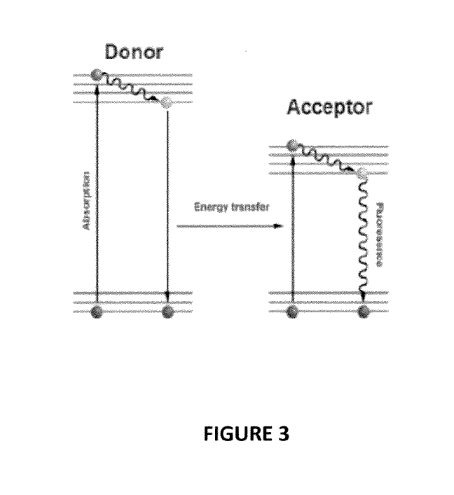

Energy transfer manifests itself through decrease or quenching of the donor emission and a reduction of excited state lifetime accompanied also by an increase in acceptor emission intensity. FIG. 3 is a Jablonski diagram that illustrates the coupled transitions involved between a donor emission and acceptor absorbance.

To enhance the energy transfer efficiency, the donor chromophore should have good abilities to absorb photons and emit photons. Furthermore, it is thought that the more overlap there is between the donor chromophore's emission spectra and the acceptor chromophore's absorption spectra, the better a donor chromophore can transfer energy to the acceptor chromophore.

In some embodiments, the biophotonic topical composition of the present disclosure further comprises an acceptor, or second, chromophore. In some embodiments, the donor, or first, chromophore has an emission spectrum that overlaps at least about 80%, about 70%, about 60%, about 50%, about 40%, about 30%, about 20%, or about 10% with an absorption spectrum of the second chromophore. In some embodiments, the first chromophore has an emission spectrum that overlaps at least about 20% with an absorption spectrum of the second chromophore. In some embodiments, the first chromophore has an emission spectrum that overlaps at least 1-10%, 5-15%, 10-20%, 15-25%, 20-30%, 25-35%, 30-40%, 35-45%, 50-60%, 55-65% or 60-70% with an absorption spectrum of the second chromophore.

The percentage (%) spectral overlap, as used herein, means the % overlap of a donor chromophore's emission wavelength range with an acceptor chromophore's absorption wavelength range, measured at spectral full width quarter maximum (FWQM). For example, FIG. 2 shows the normalized absorption and emission spectra of donor and acceptor chromophores. The spectral FWQM of the acceptor chromophore's absorption spectrum is from about 60 nm (about 515 nm to about 575 nm). The overlap of the donor chromophore's spectrum with the absorption spectrum of the acceptor chromophore is about 40 nm (from 515 nm to about 555 nm). Thus, the % overlap can be calculated as 40 nm/60 nm×100=66.6%.

In some embodiments, the second chromophore absorbs at a wavelength in the range of the visible spectrum. In some embodiments, the second chromophore has an absorption wavelength that is relatively longer than that of the first chromophore within the range of from about 50 nm to about 250 nm, from about 25 nm to about 150 nm, or from about 10 nm to about 100 nm.

As discussed above, the application of light to the compositions of the present disclosure can result in a cascade of energy transfer between the chromophores. In some embodiments, such a cascade of energy transfer provides photons that penetrate the epidermis, dermis and/or mucosa at the target tissue, including, such as, a site of wound, or a tissue afflicted with pyoderma or a skin disorder. In some embodiments, such a cascade of energy transfer is not accompanied by concomitant generation of heat. In some other embodiments, the cascade of energy transfer does not result in tissue damage.

In some embodiments, the chromophore or chromophores are selected such that their emitted fluorescent light, on photoactivation, is within one or more of the green, yellow, orange, red and infrared portions of the electromagnetic spectrum, for example having a peak wavelength within the range of from about 490 nm to about 800 nm. In some embodiments, the emitted fluorescent light has a power density of from 0.005 mW/cm² to about 10 mW/cm² or about 0.5 to about 5 mW/cm².

Suitable chromophores useful in the biophotonic topical compositions, methods, and uses of the present disclosure include, but are not limited to the following:

### Xanthene Derivatives

The xanthene derivative dyes have been used and tested for a long time worldwide. They display low toxicity and increased fluorescence. The xanthene group consists of three sub-groups: a) the fluorenes; b) fluorones; and c) the rhodoles, any of which may be suitable for the biophotonic compositions, methods, and uses of the present disclosure.

The fluorenes group comprises the pyronines (e.g., pyronine Y and B) and the rhodamines (e.g., rhodamine B. G and WT). Depending on the concentration used, both pyronines and rhodamines may be toxic and their interaction with light may lead to increased toxicity. Similar effects are known to occur for the rhodole dye group.

The fluorone group comprises the fluorescein dye and the fluorescein derivatives.

Fluorescein is a fluorophore commonly used in microscopy with an absorption maximum of 494 nm and an emission maximum of 521 nm. The disodium salt of fluorescein is known as D&C Yellow 8. It has very high fluorescence but photodegrades quickly. In the present composition, mixtures of fluorescein with other photoactivators such as indocyanin green and/or saffron red powder will confer increased photoabsorption to these other compounds.

The eosins group comprises Eosin Y (tetrabromofluorescein, acid red 87, D&C Red 22), a chromophore with an absorption maximum of 514-518 nm that stains the cytoplasm of cells, collagen, muscle fibers and red blood cells intensely red: and Eosin B (acid red 91, eosin scarlet, dibromo-dinitrofluorescein), with the same staining characteristics as Eosin Y. Eosin Y and Eosin B are collectively referred to as “Eosin,” and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light.

In some embodiments, the biophotonic composition comprises at least one of Eosin B or Eosin Y or a combination thereof in an amount of less than about 12% by weight of the total composition of. In some embodiments, at least one of Eosin B or Eosin Y or a combination thereof is present from about 0.001% to about 12%, or between about 0.01% and about 1.2%, or from about 0.01% to about 0.5%, or from about 0.01% to about 0.05%, or from about 0.1% to about 0.5%, or from about 0.5% to about 0.8% by weight of the total composition. In some embodiments, at least one of Eosin B or Eosin Y or a combination thereof is present in an amount of about 0.005% by weight of the total composition. In some embodiments, at least one of Eosin B or Eosin Y or a combination thereof is present in an amount of about 0.01% by weight of the total composition. In some embodiments, at least one of Eosin B or Eosin Y or a combination thereof is present in an amount of about 0.02% by weight of the total composition. In some embodiments, at least one of Eosin B or Eosin Y or a combination thereof is present in an amount of at about 0.05% by weight of the total composition. In some embodiments, at least one of Eosin B or Eosin Y or a combination thereof is present in an amount of about 0.1% by weight of the total composition. In some embodiments, at least one of Eosin B or Eosin Y or a combination thereof is present in an amount of about 0.2% by weight of the total composition. In some embodiments, at least one of Eosin B or Eosin Y or a combination thereof is present in an amount of at least about 0.2% by weight of the total composition but less than about 1.2% by weight of the total composition. In some embodiments, at least one of Eosin B or Eosin Y or a combination thereof is present in an amount of at least about 0.01% by weight of the total composition but less than about 12% by weight of the total composition.

In some embodiments, the biophotonic composition comprises at least one of Eosin B, Eosin Y, or a combination thereof, in an amount of less than 12% by weight of the total composition. In some embodiments, at least one of Eosin B or Eosin Y or a combination thereof is present from 0.001% to 12%, or between 0.01% and 1.2%, or from 0.01% to 0.5%, or from 0.1% to 0.5%, or from 0.5% to 0.8%, or from 0.01% to 0.05%, by weight of the total composition. In some embodiments, at least one of Eosin B or Eosin Y or a combination thereof is present in an amount of 0.005% by weight of the total composition. In some embodiments, at least one of Eosin B or Eosin Y or a combination thereof is present in an amount of 0.01% by weight of the total composition. In some embodiments, at least one of Eosin B or Eosin Y or a combination thereof is present in an amount of 0.02% by weight of the total composition. In some embodiments, at least one of Eosin B or Eosin Y or a combination thereof is present in an amount of 0.05% by weight of the total composition. In some embodiments, at least one of Eosin B or Eosin Y or a combination thereof is present in an amount of 0.1% by weight of the total composition. In some embodiments, at least one of Eosin B or Eosin Y or a combination thereof is present in an amount of 0.2% by weight of the total composition. In some embodiments, at least one of Eosin B or Eosin Y or a combination thereof is present in an amount of at least 0.2% by weight of the total composition but less than 1.2% by weight of the total composition. In some embodiments, at least one of Eosin B or Eosin Y or a combination thereof is present in an amount of at least 0.01% by weight of the total composition but less than 12% by weight of the total composition.

Phloxine B (2,4,5,7 tetrabromo 4,5,6,7,tetrachlorofluorescein, D&C Red 28, acid red 92) is a red dye derivative of fluorescein which is used for disinfection and detoxification of waste water through photooxidation. It has an absorption maximum of 535-548 nm. It is also used as an intermediate for making photosensitive dyes and drugs.

Erythrosine B, or simply Erythrosine or Erythrosin (acid red 51, tetraiodofluorescein) is a cherry-pink, coal-based fluorine food dye used as a biological stain, and a biofilm and dental plaque disclosing agent, with a maximum absorbance of 524-530 nm in aqueous solution. It is subject to photodegradation. Erythrosine is also used in some embodiments due to its photosensitivity to the light spectra used and its ability to stain biofilms. In some embodiments, the composition comprises Erythrosine B in an amount of less than about 2% by weight of total composition. In some embodiments, Erythrosine B is present in an amount from about 0.005 to about 2%, or from about 0.005% to about 1%, or about 0.01% to about 1% by weight of the total composition. In some embodiments, Erythrosine B is present in an amount of about 0.005% and about 0.15% by weight of the total composition.

Rose Bengal (4,5,6,7 tetrachloro 2,4,5,7 tetraiodofluorescein, acid red 94) is a bright bluish-pink fluorescein derivative with an absorption maximum of 544-549 nm, that has been used as a dye, biological stain and diagnostic aid. Rose Bengal is also used in synthetic chemistry to generate singlet oxygen from triplet oxygen.

Merbromine (mercurochrome) is an organo-mercuric di sodium salt of fluorescein with an absorption maximum of 508 nm. It is used as an antiseptic.

### Azo Dyes

The azo (or diazo-) dyes share the N—N group, called azo the group. They are used mainly in analytical chemistry or as food colorings and are not fluorescent. Suitable azo dyes for the compositions, methods, and uses of the disclosure include: Methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

### Biological Stains

Dye molecules commonly used in staining protocols for biological materials can also be used as photoactivators for the biophotonic compositions, methods, and uses of the disclosure. Suitable biological stains include, but are not limited to, the following:

Saffranin (Saffranin O, basic red 2) is an azo-dye and is used in histology and cytology. It is a classic counter stain in a Gram stain protocol.

Fuchsin (basic or acid) (rosaniline hydrochloride) is a magenta biological dye that can stain bacteria and has been used as an antiseptic. It has an absorption maximum of 540-555 nm.

3,3′ dihexylocarbocyanine iodide (DiOC6) is a fluorescent dye used for staining the endoplasmic reticulum, vesicle membranes and mitochondria of cells. It shows photodynamic toxicity; when exposed to blue light, has a green fluorescence.

Carminic acid (acid red 4, natural red 4) is a red glucosidal hydroxyanthrapurin naturally obtained from cochineal insects.

Indocyanin green (ICG) is used as a diagnostic aid for blood volume determination; cardiac output, or hepatic function. ICG binds strongly to red blood cells and when used in mixture with fluorescein, it increases the absorption of blue to green light.

### Carotenoids

Carotenoid dyes are also photoactivators that are suitable for use in the compositions, methods, and uses of the present disclosure.

Saffron red powder is a natural carotenoid-containing compound. Saffron is a spice derived from crocus sativus. It is characterized by a bitter taste and iodoform or hay-like fragrance; these are caused by the compounds picrocrocin and saffranal. It also contains the carotenoid dye crocin that gives its characteristic yellow-red color.

Saffron contains more than 150 different compounds, many of which are carotenoids: mangicrocin, reaxanthine, lycopene, and various a and β-carotenes, which show good absorption of light and beneficial biological activity. Also saffron can act as both a photon-transfer agent and a healing factor. Saffron color is primarily the result of a-crocin (8,8 diapo-8,8-carotenoid acid). Dry saffron red powder is highly sensitive to fluctuating pH levels and rapidly breaks down chemically in the presence of light and oxidizing agents. It is more resistant to heat. Data show that saffron has anticarcinogenic, immunomodulating and antioxidant properties. For absorbance, the crocin specific photon wavelength is 440 nm (blue light). It has a deep red colour and forms crystals with a melting point of 186° C. When dissolved in water, it forms an orange solution.

Crocetin, another compound of saffron, was found to express an antilipidemic action and promote oxygen penetration in different tissues. More specifically, an increased oxygenation of the endothelial cells of the capillaries was observed. Additionally, an increase of the oxygenation of muscles and cerebral cortex was observed and led to an improved survival rate in laboratory animals with induced hemorrhagic shock or emphysema.

Anatto, a spice, contains as main constituent (70-80%) the carotenoid bixin which displays relevant antioxidative properties. β-carotene, also displays suitable characteristics.

Fucoxanthine is a constituent of brown algae with a pronounced ability for photosensitization of redox reactions.

### Chlorophyll Dyes

Exemplary chlorophyll dyes that are useful in the compositions, methods, and uses of the disclosure, include, but are not limited to, chlorophyll a, chlorophyll b, oil soluble chlorophyll, bacteriochlorophyll a, bacteriochlorophyll b, bacteriochlorophyll c, bacteriochlorophyll d, protochlorophyll, protochlorophyll a, amphiphilic chlorophyll derivative 1, and amphiphilic chlorophyll derivative 2.

In some aspects of the disclosure, the one or more chromophores of the biophotonic composition disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin. Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Saffranin O), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Celestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta 1, Magenta 11, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow 5, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

Chromophores can be selected, for example, on their emission wavelength properties in the case of fluorophores, on the basis of their energy transfer potential, their ability to generate reactive oxygen species, or their antimicrobial effect.

In some embodiments, the biophotonic composition comprises Eosin Y as a first chromophore. In some embodiments, the biophotonic composition comprises Eosin Y as a first chromophore and any one or more of Rose Bengal, Erythrosin, Phloxine B as a second chromophore. It is believed that these combinations have a synergistic effect as Eosin Y can transfer energy to Rose Bengal, Erythrosin or Phloxine B when activated. This transferred energy is then emitted as fluorescence or by production of reactive oxygen species. This absorbed and re-emitted light is thought to be transmitted throughout the composition, and also to be transmitted into the site of treatment.

In some embodiments, the biophotonic composition of the present disclosure comprises the following synergistic combinations: Eosin Y and Fluorescein; Fluorescein and Rose Bengal; Erythrosine in combination with one of more of Eosin Y, Rose Bengal or Fluorescein; or Phloxine B in combination with one or more of Eosin Y, Rose Bengal, Fluorescein and Erythrosine. Other synergistic chromophore combinations are also contemplated herein.

By means of synergistic effects of the chromophore combinations in the composition, chromophores which cannot normally be activated by an activating light (such as a blue light from an LED) can be activated through energy transfer from chromophores which are activated by the activating light. In this way, the different properties of photoactivated chromophores can be harnessed and tailored according to the cosmetic or the medical therapy required.

For example, Rose Bengal can generate a high yield of singlet oxygen when photoactivated in the presence of molecular oxygen, however it has a low quantum yield in terms of emitted fluorescent light. Rose Bengal has a peak absorption around 540 nm; so it is normally activated by green light. Eosin Y has a high quantum yield and can be activated by blue light. By combining Rose Bengal with Eosin Y, one obtains a composition which can emit therapeutic fluorescent light and generate singlet oxygen when activated by blue light. In this case, the blue light photoactivates Eosin Y, which transfers some of its energy to Rose Bengal and emits some energy as fluorescence.

Chromophore combinations can also have a synergistic effect in terms of their photoactivated state. In some embodiments, two chromophores may be used, one of which emits fluorescent light when activated in the blue and green range, and the other which emits fluorescent light in the red, orange and yellow range, thereby complementing each other and irradiating the target tissue with a broad wavelength of light having different depths of penetration into target tissue and different therapeutic effects.

### Healing Factors

In some embodiments, the biophotonic compositions of the present disclosure further comprise one or more healing factors. Healing factors comprise compounds that promote or enhance the healing or regenerative process of the tissues on the application site of the composition. During the photoactivation of the composition, there is an increase of the absorption of molecules at the treatment site. An augmentation in the blood flow at the site of treatment is observed for an extended period of time. An increase in the lymphatic drainage and a possible change in the osmotic equilibrium due to the dynamic interaction of the free radical cascades can be enhanced or even fortified with the inclusion of healing factors.

In some embodiments, the biophotonic compositions of this disclosure comprises one or more healing factors selected from, but not limited to, hyaluronic acid, glucosamine, allantoin, or saffron.

Suitable healing factors for the compositions, methods and uses of the present disclosure include, but are not limited to, the following:

### Hyaluronic Acid (Hyaluronan or Hyaluronate)

Hyaluronic acid (hyaluronan or hyaluronate) is a non-sulfated glycosaminoglycan, distributed widely throughout connective, epithelial and neural tissues. It is one of the primary components of the extracellular matrix, and contributes significantly to cell proliferation and migration. Hyaluronan is a major component of the skin, where it is involved in tissue repair. While it is abundant in extracellular matrices, it contributes to tissue hydrodynamics, movement and proliferation of cells and participates in a wide number of cell surface receptor interactions, notably those including primary receptor CD44. The hyaluronidase enzymes degrade hyaluronan and there are at least seven types of hyaluronidase-like enzymes in humans, several of which are tumor suppressors. The degradation products of hyaluronic acid, the oligosaccharides and the very-low molecular weight hyaluronic acid, exhibit pro-angiogenic properties. In addition, recent studies show that hyaluronan fragments, but not the native high molecular mass of hyaluronan, can induce inflammatory responses in macrophages and dendritic cells in tissue injury. Hyaluronic acid is well suited to biological applications targeting the skin. Due to its high biocompatibility, it is used to stimulate tissue regeneration. Current studies evidenced hyaluronic acid appearing in the early stages of healing to physically create room for white blood cells that mediate the immune response. It is used in the synthesis of biological scaffolds for wound healing applications and in wrinkle treatment. In certain embodiments, the composition comprises hyaluronic acid in an amount of less than about 2% by weight of the total composition. In some embodiments, hyaluronic acid is present in an amount from about 0.001% to about 2%, from about 0.002% to about 2%, or from about 0.002% to about 1% by weight of the total composition. In some embodiments, hyaluronic acid is not present (0%) in the biophotonic compositions of this disclosure.

### Glucosamine

Glucosamine is one of the most abundant monosaccharides in human tissues and a precursor in the biological synthesis of glycosylated proteins and lipids. It is commonly used in the treatment of osteoarthritis. The common form of glucosamine used is its sulfate salt. Glucosamine shows a number of effects including, anti-inflammatory activity, stimulation of the synthesis of proteoglycans and the synthesis of proteolytic enzymes. A suitable range of concentration over which glucosamine can be used in the present composition is from less than about 5% by weight of the total composition. In some embodiments, glucosamine is present in an amount from about 0.0001% to about 5%, from about 0.0001% to about 3%, from about 0.001% to about 3%, from about 0.001% to about 1%, from about 0.01% to about 1%, or from about 1% to about 3% by weight of the total composition.

### Allantoin

Allantoin is a diureide of glyosilic acid. It has keratolytic effect, increases the water content of the extracellular matrix, enhances the desquamation of the upper layers of dead (apoptotic) skin cells, and promotes skin proliferation and wound healing. In certain embodiments, the composition comprises allantoin in an amount of less than about 1% by weight of the total composition. In some embodiments, allantoin is present in an amount from about 0.001% to about 1%, from about 0.002% to about 1%, from about 0.02% to about 1%, or from about 0.02% to about 0.5% by weight of the total composition.

Also, saffron can act as both a photon-transfer agent and a healing factor.

### Chelating Agents

In some embodiments, the biophotonic compositions of the present disclosure comprise one or more chelating agents. Chelating agents can be included to promote smear layer removal in closed pockets and difficult to reach lesions. Chelating agents act as a metal ion quencher and as a buffer. In some embodiments, the biophotonic compositions of this disclosure comprise one or more chelating agents selected from, but are not limited to, ethylenediaminetetraacetic acid or ethylene glycol tetraacetic acid. Suitable chelating agents for the compositions, methods and uses of the disclosure include, but are not limited to:

### Ethylenediaminetetraacetic Acid (EDTA)

Ethylenediaminetetraacetic acid (EDTA) is an amino acid and is used to sequester di- and trivalent metal ions. EDTA binds to metals via four carboxylate and two amine groups. EDTA forms especially strong complexes with Mn(III), Fe(III), Cu(III), Co(III). It is used to buffer solutions.

### Ethylene Glycol Tetraacetic Acid (EGTA)

Ethylene glycol tetraacetic acid (EGTA) is related to EDTA, but with a much higher affinity for calcium than magnesium ions. It is useful for making buffer solutions that resemble the environment inside living cells.

### Gelling Agents

In some embodiments, the biophotonic compositions of the present disclosure comprise one or more gelling agents. The gelling agent may be an agent capable of forming a cross-linked matrix, including physical and/or chemical cross-links. The gelling agent can be biocompatible, and may be biodegradable. In some embodiments, the gelling agent is able to form a hydrogel or a hydrocolloid. An appropriate gelling agent is one that can form a viscous liquid or a semisolid. In some embodiments, the gelling agent and/or the composition has appropriate light transmission properties. It is also important to select a gelling agent which will allow biophotonic activity of the chromophore(s). For example, some chromophores require a hydrated environment in order to fluoresce. The gelling agent may be able to form a gel by itself or in combination with other ingredients such as water or another gelling agent, or when applied to a treatment site, or when illuminated with light.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing; and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); silicones, polyvinyl silicates, tetramethoxyorthosilicates, methyltrimethoxyorthosilicates, tetraalkoxyorthosilicates, trialkoxyorthosilicates, pressure sensitive silicone adhesives (such as BioPSA from Dow-Corning), and polyvinylamines.

The gelling agent according to some embodiments of the present disclosure may include a polymer selected from any of synthetic or semi-synthetic polymeric materials; polyacrylate copolymers; cellulose derivatives and polymethyl vinyl ether/maleic anhydride copolymers. In some embodiments, the hydrophilic polymer comprises a polymer that is a high molecular weight (i.e.; molar masses of more than about 5,000, and in some instances, more than about 10,000, or about 100,000, or about 1,000,000) and/or cross-linked polyacrylic acid polymer.

In some embodiments, the gelling agent comprises a carbomer. Carbomers are synthetic high molecular weight polymer of acrylic acid that are cross-linked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. Aqueous solution of Carbopol® is acidic in nature due to the presence of free carboxylic acid residues. Neutralization of this solution cross-links and gelatinizes the polymer to form a viscous integral structure of desired viscosity.

Carbomers are available as fine white powders which disperse in water to form acidic colloidal suspensions (a 1% dispersion has a pH of approximately 3) of low viscosity. Neutralization of these suspensions using a base, for example sodium, potassium or ammonium hydroxides, low molecular weight amines and alkanolamines, results in the formation of translucent gels. Nicotine salts such as nicotine chloride form stable water-soluble complexes with carbomers at about pH 3.5 and are stabilized at an optimal pH of about 5.6.

In some embodiments of the disclosure, the carbomer is Carbopol®. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like. Carbopols are versatile controlled-release polymers, as described by Brock (Pharmacotherapy, 14:430-7 (1994), incorporated herein by reference) and Durrani (Pharmaceutical Res. (Supp.) 8:S-135 (1991), incorporated herein by reference), and belong to a family of carbomers which are synthetic, high molecular weight, non-linear polymers of acrylic acid, crosslinked with polyalkenyl polyether. In some embodiments, the carbomer is Carbopol® 974P NF. 980 NF, 5984 EP, ETD 2020NF, Ultrez 10 NF, 934 NF, 934P NF or 940 NF. In some embodiments, the carbomer is Carbopol® 980 NF, ETD 2020 NF, Ultrez 10 NF, Ultrez 21 or 1382 Polymer, 1342 NF, 940 NF. In some embodiments, about 0.05% to about 10%, about 0.5% to about 5%, or about 1% to about 3% by weight of the total composition of a high molecular weight carbopol can be present as the gelling agent. In some embodiments, the biophotonic composition of the disclosure comprises about from 0.05% to about 10%, from about 0.5% to about 5%, or from about 1% to about 3% by weight of the total composition of a high molecular weight carbopol.

In some embodiments, the gelling agent comprises a hygroscopic and/or a hydrophilic material useful for their water attracting properties. The hygroscopic or hydrophilic material may include, but is not limited to, glucosamine, glucosamine sulfate, polysaccharides, cellulose derivatives (hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like), glycosaminoglycan, poly(vinyl alcohol), poly(2-hydroxyethylmethylacrylate), polyethylene oxide, collagen, chitosan, alginate, a poly(acrylonitrile)-based hydrogel, poly(ethylene glycol)/poly(acrylic acid) interpenetrating polymer network hydrogel, polyethylene oxide-polybutylene terephthalate, hyaluronic acid, high-molecular-weight polyacrylic acid, poly(hydroxy ethylmethacrylate), poly(ethylene glycol), tetraethylene glycol diacrylate, polyethylene glycol methacrylate, and poly(methyl acrylate-co-hydroxyethyl acrylate). In some embodiments, the hydrophilic gelling agent is selected from glucose, modified starch, methyl cellulose, carboxymethyl cellulose, propyl cellulose, hydroxypropyl cellulose, carbomers, alginic acid, sodium alginate, potassium alginate, ammonium alginate, calcium alginate, agar, carrageenan, locust bean gum, pectin, and gelatin.

The gelling agent may be protein-based/naturally derived material such as sodium hyaluronate, gelatin or collagen, lipids, or the like. The gelling agent may be a polysaccharide such as starch, chitosan, chitin, agarose, agar, locust bean gum, carrageenan, gellan gum, pectin, alginate, xanthan, guar gum, and the like.

In some embodiments, the biophotonic composition of the present disclosure can include up to about 2% by weight of the total composition of sodium hyaluronate as the single gelling agent. In some embodiments, the biophotonic composition can include more than about 4% or more than about 5% by weight of the total composition of gelatin as the single gelling agent. In some embodiments, the biophotonic composition can include up to about 10% or up to about 8% starch as the single gelling agent. In some embodiments, the biophotonic composition can include more than about 5% or more than about 10% by weight of the total composition of collagen as the gelling agent. In some embodiments, about 0.1% to about 10% or about 0.5% to about 3% by weight of the total composition of chitin can be used as the gelling agent. In some embodiments, about 0.5% to about 5% by weight of the total composition of corn starch or about 5% to about 10% by weight of the total composition of corn starch can be used as the gelling agent. In some embodiments, more than about 2.5 wt % by weight of the total composition of alginate can be used in the composition as the gelling agent. In some embodiments, the percentages by weight percent of the total composition of the gelling agents can be as follows: cellulose gel (from about 0.3% to about 2.0%), konjac gum (from about 0.5% to about 0.7%), carrageenan gum (from about 0.02% to about 2.0%), xanthan gum (from about 0.01% to about 2.0%), acacia gum (from about 3% to about 30%), agar (from about 0.04% to about 1.2%), guar gum (from about 0.1% to about 1%), locust bean gum (from about 0.15% to about 0.75%), pectin (from about 0.1% to about 0.6%), tara gum (from about 0.1% to about 1.0%), polyvinylpyrrolidone (from about 1% to about 5%), sodium polyacrylate (from about 1% to about 10%). Other gelling agents can be used in amounts sufficient to gel the composition or to sufficiently thicken the composition. It will be appreciated that lower amounts of the above gelling agents may be used in the presence of another gelling agent or a thickener.

In some embodiments, the biophotonic composition of the present disclosure may be further encapsulated, e.g., in a membrane. Such a membrane may be transparent, and/or substantially, or fully impermeable. The membrane may be impermeable to liquid but permeable to gases such as air. In some embodiments, the composition may form a membrane that encapsulates the chromophore(s) of the biophotonic topical composition, where the membrane may be substantially impermeable to liquid and/or gas. The membrane may be formed of one or more lipidic agents, polymers, gelatin, cellulose or cyclodextrins, or the like. In some embodiments, the membrane is translucent or transparent to allow light to infiltrate to and from the chromophore(s). In some embodiments, the composition is a dendrimer with an outer membrane comprising poly(propylene amine). In some embodiments, the outer membrane comprises gelatin.

### Polyols

According to some embodiments, the biophotonic compositions of the present disclosure may optionally comprise one or more polyols. Suitable polyols that may be included in the composition include, but are not limited to a diol, a triol, a saccharide, glycerine, butane-1,2,3-triol, butane-1,2,4-triol, hexane-1,2,6-triol, propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol. In some embodiments when the biophotonic composition of the disclosure includes one or more polyols, the polyol is glycerine. In some embodiments when the biophotonic composition of the disclosure includes one or more polyols, the polyol is propylene glycol. In some embodiments when the biophotonic composition of the disclosure includes one or more polyols, the polyol is a combination of glycerine and propylene glycol.

In some embodiments, one or more polyols are present in an amount of from about 5% to about 75% by weight of the total composition, such as from about 5% to about 75% by weight of the total composition. In some embodiments, one or more polyols are present in an amount of from about 10% to about 75% by weight of the total composition (e.g., from 10% to 75%) from by weight of the total composition. In some embodiments, one or more polyols are present in an amount of about 15% to about 75% (e.g., from 15% to 75%) by weight of the total composition. In some embodiments, one or more polyols are present in an amount of about 20% to about 75% (e.g., from 20% to 75%) by weight of the total composition.

### Antimicrobials

According to some embodiments, the biophotonic compositions of the present disclosure may optionally comprise one or more antimicrobials. Antimicrobials kill microbes or inhibit their growth or accumulation. Exemplary antimicrobials (or antimicrobial agent) are recited in U.S. Patent Application Publication Nos. 20040009227 and 20110081530, the contents of which are incorporated herein by reference. Suitable antimicrobials for use in the methods of the present disclosure include, but not limited to, phenolic and chlorinated phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

Specific phenolic and chlorinated phenolic antimicrobial agents that can be used in the disclosure include, but are not limited to: phenol; 2-methyl phenol; 3-methyl phenol; 4-methyl phenol; 4-ethyl phenol; 2,4-dimethyl phenol; 2,5-dimethyl phenol; 3,4-dimethyl phenol; 2,6-dimethyl phenol; 4-n-propyl phenol; 4-n-butyl phenol; 4-n-amyl phenol; 4-tert-amyl phenol; 4-n-hexyl phenol; 4-n-heptyl phenol; mono- and poly-alkyl and aromatic halophenols; p-chlorophenyl; methyl p-chlorophenol; ethyl p-chlorophenol; n-propyl p-chlorophenol; n-butyl p-chlorophenol; n-amyl p-chlorophenol; sec-amyl p-chlorophenol; n-hexyl p-chlorophenol; cyclohexyl p-chlorophenol; n-heptyl p-chlorophenol; n-octyl; p-chlorophenol; o-chlorophenol; methyl o-chlorophenol; ethyl o-chlorophenol; n-propyl o-chlorophenol; n-butyl o-chlorophenol; n-amyl o-chlorophenol; tert-amyl o-chlorophenol; n-hexyl o-chlorophenol; n-heptyl o-chlorophenol; o-benzyl p-chlorophenol; o-benxyl-m-methyl p-chlorophenol; o-benzyl-m,m-dimethyl p-chlorophenol; o-phenyl ethyl p-chlorophenol; o-phenylethyl-m-methyl p-chlorophenol; 3-methyl p-chlorophenol 3,5-dimethyl p-chlorophenol, 6-ethyl-3-methyl p-chlorophenol, 6-n-propyl-3-methyl p-chlorophenol; 6-iso-propyl-3-methyl p-chlorophenol; 2-ethyl-3,5-dimethyl p-chlorophenol; 6-sec-butyl-3-methyl p-chlorophenol; 2-iso-propyl-3,5-dimethyl p-chlorophenol; 6-diethylmethyl-3-methyl p-chlorophenol; 6-iso-propyl-2-ethyl-3-methyl p-chlorophenol; 2-sec-amyl-3,5-dimethyl p-chlorophenol; 2-diethylmethyl-3,5-dimethyl p-chlorophenol; 6-sec-octyl-3-methyl p-chlorophenol; p-chloro-m-cresol p-bromophenol; methyl p-bromophenol; ethyl p-bromophenol; n-propyl p-bromophenol; n-butyl p-bromophenol; n-amyl p-bromophenol; sec-amyl p-bromophenol; n-hexyl p-bromophenol; cyclohexyl p-bromophenol; o-bromophenol; tert-amyl o-bromophenol; n-hexyl o-bromophenol; n-propyl-m,m-dimethyl o-bromophenol; 2-phenyl phenol; 4-chloro-2-methyl phenol; 4-chloro-3-methyl phenol; 4-chloro-3,5-dimethyl phenol; 2,4-dichloro-3,5-dimethylphenol; 3,4,5,6-tetabromo-2-methylphenol; 5-methyl-2-pentylphenol; 4-isopropyl-3-methylphenol; para-chloro-metaxylenol (PCMX); chlorothymol; phenoxyethanol; phenoxyisopropanol; and 5-chloro-2-hydroxydiphenylmethane.

Resorcinol and its derivatives can also be used as antimicrobial agents. Specific resorcinol derivatives include, but are not limited to: methyl resorcinol; ethyl resorcinol; n-propyl resorcinol; n-butyl resorcinol; n-amyl resorcinol; n-hexyl resorcinol; n-heptyl resorcinol; n-octyl resorcinol; n-nonyl resorcinol; phenyl resorcinol; benzyl resorcinol; phenylethyl resorcinol; phenylpropyl resorcinol; p-chlorobenzyl resorcinol; 5-chloro-2,4-dihydroxydiphenyl methane; 4′-chloro-2,4-dihydroxydiphenyl methane; 5-bromo-2,4-dihydroxydiphenyl methane; and 4′-bromo-2,4-dihydroxydiphenyl methane.

Specific bisphenolic antimicrobial agents that can be used in the disclosure include, but are not limited to: 2,2′-methylene bis-(4-chlorophenol); 2,4,4′trichloro-2′-hydroxy-diphenyl ether, which is sold by Ciba Geigy, Florham Park, N.J. under the trade name Triclosan®; 2,2′-methylene bis-(3,4,6-trichlorophenol); 2,2′-methylene bis-(4-chloro-6-bromophenol); bis-(2-hydroxy-3,5-dichlorophenyl)sulphide; and bis-(2-hydroxy-5-chlorobenzyl)sulphide.

Specific benzoic esters (parabens) that can be used in the disclosure include, but are not limited to: methylparaben; propylparaben; butylparaben; ethylparaben; isopropylparaben; isobutylparaben; benzylparaben; sodium methylparaben; and sodium propylparaben.

Specific halogenated carbanilides that can be used in the disclosure include, but are not limited to: 3,4,4′-trichlorocarbanilides, such as 3-(4-chlorophenyl)-1-(3,4-dichlorphenyl)urea sold under the tradename Triclocarban® by Ciba-Geigy, Florham Park, N.J.; 3-trifluoromethyl-4,4′-dichlorocarbanilide; and 3,3′,4-trichlorocarbanilide.

Specific polymeric antimicrobial agents that can be used in the disclosure include, but are not limited to: polyhexamethylene biguanide hydrochloride; and poly(iminoimidocarbonyl iminoimidocarbonyl iminohexamethylene hydrochloride), which is sold under the tradename Vantocil® IB.

Specific thazolines that can be used in the disclosure include, but are not limited to that sold under the tradename Micro-Check®; and 2-n-octyl-4-isothiazolin-3-one, which is sold under the tradename Vinyzene® IT-3000 DIDP.

Specific trichloromethylthioimides that can be used in the disclosure include, but are not limited to: N-(trichloromethylthio)phthalimide, which is sold under the tradename Fungitrol®; and N-trichloromethylthio-4-cyclohexene-1,2-dicarboximide, which is sold under the tradename Vancide®.

Specific natural antimicrobial agents that can be used in the disclosure include, but are not limited to, oils of: anise, lemon, orange, rosemary, wintergreen, thyme, lavender, cloves, hops, tea tree, citronella, wheat, barley, lemongrass, cedar leaf, cedarwood, cinnamon, fleagrass, geranium, sandalwood, violet, cranberry, eucalyptus, vervain, peppermint, gum benzoin, basil, honey, fennel, fir, balsam, menthol, ocmea origanuin, hydastis, carradensis, Berberidaceac daceae, Ratanhiae longa, and Curcuma longa. Also included in this class of natural antimicrobial agents are the key chemical components of the plant oils which have been found to provide antimicrobial benefit. These chemicals include, but are not limited to: anethol, catechole, camphene, thymol, eugenol, eucalyptol, ferulic acid, famesol, hinokitiol, tropolone, limonene, menthol, methyl salicylate, carvacol, terpineol, verbenone, berberine, ratanhiae extract, caryophellene oxide, citronellic acid, curcumin, nerolidol, and geraniol.

Specific metal salts that can be used in the disclosure include, but are not limited to, salts of metals in Groups 3a-5a, 3b-7b, and 8 of the periodic table. Specific examples of metal salts include, but are not limited to, salts of: aluminum, zirconium, zinc, silver, gold, copper, lanthanum, tin, mercury, bismuth, selenium, strontium, scandium, yttrium, cerium, praseodymiun, neodymium, promethum, samarium, europium, gadolinium, terbium, dysprosium, holmium, erbium, thalium, ytterbium, lutetium, and mixtures thereof. An example of the metal-ion based antimicrobial agent is sold under the tradename HealthShield®, and is manufactured by HealthShield Technology, Wakefield, Mass.

Specific broad-spectrum antimicrobial agents that can be used in the disclosure include, but are not limited to, those that are recited in other categories of antimicrobial agents herein.

Additional antimicrobial agents that can be used in the methods of the disclosure include, but are not limited to: pyrithiones, and in particular pyrithione-including zinc complexes such as these sold under the tradename Octopirox®; dimethyidimethylol hydantoin, which is sold under the tradename Glydant®; methylchloroisothiazolinone/methylisothiazolinone, which is sold under the tradename Kathon CG®; sodium sulfite; sodium bisulfate; imidazolidinyl urea, which is sold under the tradename Germall 115®; diazolidinyl urea, which is sold under the tradename Germall 11®; benzyl alcohol v2-bromo-2-nitropropane-1,3-diol, which is sold under the tradename Bronopol®; formalin or formaldehyde; iodopropenyl butylcarbamate, which is sold under the tradename Polyphase P100®; chloroacetamide; methanamine; methyldibromonitrile glutaronitrile (1,2-dibromo-2,4-dicyanobutane), which is sold under the tradename Tektamer®; glutaraldehyde; 5-bromo-5-nitro-1,3-dioxane, which is sold under the tradename Bronidox®; phenethyl alcohol; o-phenylphenol/sodium o-phenylphenol sodium hydroxymethylglycinate, which is sold under the tradename Suttocide A®; polymethoxy bicyclic oxazolidine; which is sold under the tradename Nuosept C®; dimethoxane; thimersal; dichlorobenzyl alcohol; captan; chlorphenenesin; dichlorophene; chlorbutanol; glyceryl laurate, halogenated diphenyl ethers; 2,4,4′-trichloro-2′-hydroxy-diphenyl ether, which is sold under the tradename Triclosan® and is available from Ciba-Geigy, Florham Park, N.J.; and 2,2′-dihydroxy-5,5′-dibromo-diphenyl ether.

Additional antimicrobial agents that can be used in the methods of the disclosure include those disclosed by U.S. Pat. Nos. 3,141,321; 4,402,959; 4,430,381; 4,533,435; 4,625,026; 4,736,467; 4,855,139; 5,069,907; 5,091,102; 5,639,464; 5,853,883; 5,854,147; 5,894,042; and 5,919,554, and U.S. Pat. Appl. Publ. Nos. 20040009227 and 20110081530, the contents of all of which are incorporated herein by reference.

Collagens and Agents that Promote Collagen Synthesis

According to some embodiments, the biophotonic compositions of the present disclosure may optionally comprise one or more collagens and/or agents that promote collagen synthesis. Collagen is a fibrous protein produced in dermal fibroblast cells and forming 70% of the dermis and benefits all stages of the wound healing process. Thus, collagens and agents that promote collagen synthesis may also be useful in the present disclosure. Agents that promote collagen synthesis (i.e., pro-collagen synthesis agents) include amino acids, peptides, proteins, lipids, small chemical molecules, natural products and extracts from natural products.

For instance, it was discovered that intake of vitamin C, iron, and collagen can effectively increase the amount of collagen in skin or bone. See, e.g., U.S. Patent Application Publication 20090069217, the contents of which are all incorporated herein by reference. Examples of the vitamin C include an ascorbic acid derivative such as L-ascorbic acid or sodium L-ascorbate, an ascorbic acid preparation obtained by coating ascorbic acid with an emulsifier or the like, and a mixture containing two or more of those vitamin Cs at an arbitrary rate. In addition, natural products containing vitamin C such as acerola and lemon may also be used. Examples of the iron preparation include: an inorganic iron such as ferrous sulfate, sodium ferrous citrate, or ferric pyrophosphate; an organic iron such as heme iron, ferritin iron, or lactoferrin iron; and a mixture containing two or more of those irons at an arbitrary rate. In addition, natural products containing iron such as spinach or liver may also be used. Moreover, examples of the collagen include: an extract obtained by treating bone, skin, or the like of a mammal such as bovine or swine with an acid or alkaline; a peptide obtained by hydrolyzing the extract with a protease such as pepsin, trypsin, or chymotrypsin; and a mixture containing two or more of those collagens at an arbitrary rate. Collagens extracted from plant sources may also be used.

Additional pro-collagen synthesis agents are described, for example, in U.S. Pat. Nos. 7,598,291, 7,722,904, 6,203,805, 5,529,769, etc, and U.S. Patent Application Publications 20060247313, 20080108681, 20110130459, 20090325885, and 20110086060, the contents of all of which are incorporated herein by reference.

### Additional Components

In some embodiments, the compositions, methods, and uses of the disclosure may further comprise ingredients such as humectants (e.g., glycerine, ethylene glycol, and propylene glycol), preservatives such as parabens, and pH adjusters such as sodium hydroxide, sodium bicarbonate, and HCl. In some embodiments, the pH of the composition is within or adjusted to the range of from about 4 to about 10. In some embodiments, the pH of the composition is in or adjusted to the range of about 4 to about 9. In some embodiments, the pH of the composition is in or adjusted to the range of about 4 to about 8. In some embodiments, the pH of the composition is within the range of about 4 to about 7. In some embodiments, the pH of the composition is within the range of about 4 to about 6.5. In some embodiments, the pH of the composition is within the range of about 4 to about 6. In some embodiments, the pH of the composition is within the range of about 4 to about 5.5. In some embodiments, the pH of the composition is within the range of about 4 to about 5. In some embodiments, the pH of the composition is within the range of about 5.0 to about 8.0. In some embodiments, the pH of the composition is within the range of about 6.0 to about 8.0. In some embodiments, the pH of the composition is within the range of about 6.5 to about 7.5. In some embodiments, the pH of the composition is within the range of about 5.5 to about 7.5.

In some embodiments, the pH of the composition is in or adjusted to the range of 4 to 10. In some embodiments, the pH of the composition is in or adjusted to the range of 4 to 9. In some embodiments, the pH of the composition is in or adjusted to the range of 4 to 8. In some embodiments, the pH of the composition is within the range of 4 to 7. In some embodiments, the pH of the composition is within the range of 4 to 6.5. In some embodiments, the pH of the composition is within the range of 4 to 6. In some embodiments, the pH of the composition is within the range of 4 to 5.5. In some embodiments, the pH of the composition is within the range of 4 to 5. In some embodiments, the pH of the composition is within the range of 5.0 to 8.0. In some embodiments, the pH of the composition is within the range of 6.0 to 8.0. In some embodiments, the pH of the composition is within the range of 6.5 to 7.5. In some embodiments, the pH of the composition is within the range of 5.5 to 7.5.

In some embodiments, the biophotonic compositions of the disclosure may further comprise an aqueous substance (water) or an alcohol. Alcohols include, but are not limited to, ethanol, propanol, isopropanol, butanol, iso-butanol, t-butanol or pentanol. In some embodiments, the chromophore or combination of chromophores is in solution in a medium of the biophotonic composition. In some embodiments, the chromophore or combination of chromophores is in solution in a medium of the biophotonic composition, wherein the medium is an aqueous substance.

### Methods of Use and Treatment

### Photoactivation

The biophotonic compositions suitable for use in the methods of the present disclosure may be selected from any of the embodiments of the biophotonic compositions described above. For instance, the biophotonic compositions useful in the method of the present disclosure may comprise a chromophore that undergoes at least partial photobleaching upon application of light. The chromophore may absorb at a wavelength of from about 200 nm to about 800 nm, such as, from about 200 nm to about 700 nm, from about 200 nm to about 600 nm, or from about 200 nm to about 500 nm. In some embodiments, the chromophore absorbs at a wavelength of from about 200 nm to about 600 nm. In some embodiments, the chromophore absorbs light at a wavelength of from about 200 nm to about 300 nm, from about 250 nm to about 350 nm, from about 300 nm to about 400 nm, from about 350 nm to about 450 nm, from about 400 nm to about 500 nm, from about 450 nm to about 650 nm, from about 600 nm to about 700 nm, from about 650 nm to about 750 nm, or from about 700 nm to about 800 nm. In some embodiments, suitable biophotonic compositions for the methods of the present disclosure may further comprise at least one additional chromophore (e.g., a second chromophore). The absorption spectrum of the second chromophore overlaps at least about 80%, about 70%, about 60%, about 50%, about 40%, about 30%, or about 20% with the emission spectrum of the first chromophore. In some embodiments, the first chromophore has an emission spectrum that overlaps at least 1-10%, 5-15%, 10-20%, 15-25%, 20-30%, 25-35%, 30-40%, 35-45%, 50-60%, 55-65% or 60-70% with an absorption spectrum of the second chromophore.

Illumination of the biophotonic composition with light may cause a transfer of energy from the first chromophore to the second chromophore. Subsequently, the second chromophore may emit energy as fluorescence and/or generate reactive oxygen species. In some embodiments of the methods the present disclosure, energy transfer caused by the application of light is not accompanied by concomitant generation of heat, or does not result in tissue damage.

In the methods of the present disclosure, any source of actinic light can be used to illuminate the biophotonic compositions. Any type of halogen, LED or plasma arc lamp or laser may be suitable. The primary characteristic of suitable sources of actinic light will be that they emit light in a wavelength (or wavelengths) appropriate for activating the one or more photoactivators present in the composition. In some embodiments, an argon laser is used. In some embodiments, a potassium-titanyl phosphate (KTP) laser (e.g., a GreenLight™ laser) is used. In another embodiment, sunlight may be used. In some embodiments, a LED photocuring device is the source of the actinic light, such as a LED lamp (e.g., a Thera™ lamp). In some embodiments, the source of the actinic light is a source of light having a wavelength between about 200 nm and about 800 nm. In some embodiments, the source of the actinic light is a source of visible light having a wavelength between about 400 nm and about 700 nm. In some embodiments, the source of the actinic light is a source of visible light having a wavelength between about 400 nm and about 600 nm. In some embodiments, the source of the actinic light is a source of visible light having a wavelength between about 400 nm and about 550 nm. In some embodiments, the source of the actinic light is a source of visible light having a wavelength between about 380 nm and about 700 nm. In some embodiments, the source of the actinic light is a source of visible light having a wavelength between about 380 nm and about 600 nm. In some embodiments, the source of the actinic light is a source of visible light having a wavelength between about 380 nm and about 550 nm.

In some embodiments, the source of the actinic light is a source of light having a wavelength between 200 nm and 800 nm. In some embodiments, the source of the actinic light is a source of visible light having a wavelength between 400 nm and 700 nm. In some embodiments, the source of the actinic light is a source of visible light having a wavelength between 400 nm and 600 nm. In some embodiments, the source of the actinic light is a source of visible light having a wavelength between 400 nm and 550 nm. In some embodiments, the source of the actinic light is a source of visible light having a wavelength between 380 nm and 700 nm. In some embodiments, the source of the actinic light is a source of visible light having a wavelength between 380 nm and 600 nm. In some embodiments, the source of the actinic light is a source of visible light having a wavelength between 380 nm and 550 nm.

In some embodiments, the biophotonic composition of the disclosure is illuminated with violet and/or blue light. Furthermore, the source of actinic light should have a suitable power density. Suitable power density for non-collimated light sources (LED, halogen or plasma lamps) are in the range from about 1 mW/cm² to about 200 mW/cm². Suitable power density for laser light sources is in the range from about 0.5 mW/cm² to about 0.8 mW/cm².

In some embodiments of the methods of the present disclosure, the light has an energy at the patient's skin of from about 1 mW/cm² to about 500 mW/cm², from about 1 to about 300 mW/cm², or from about 1 to about 200 mW/cm², wherein the energy applied depends at least on the condition being treated, the wavelength of the light, the distance of the patient's skin from the light source, and the thickness of the biophotonic composition. In some embodiments, the light at the patient's skin is from about 1 to about 40 mW/cm², or from about 20 to about 60 mW/cm², or from about 40 to about 80 mW/cm², or from about 60 to about 100 mW/cm², or from about 80 to about 120 mW/cm², or from about 100 to about 140 mW/cm², or from about 120 to about 160 mW/cm², or from about 140 to about 180 mW/cm², or from about 160 to about 200 mW/cm², or from about 110 to about 240 mW/cm², or from about 110 to about 150 mW/cm², or from about 190 to about 240 mW/cm².

In some embodiments, a mobile device can be used to activate embodiments of the biophotonic compositions of the present disclosure, wherein the mobile device can emit light having an emission spectrum which overlaps an absorption spectrum of the chromophore in the biophotonic composition. The mobile device can have a display screen through which the light is emitted and/or the mobile device can emit light from a flashlight which photoactivates the biophotonic compositions.

In some embodiments, a display screen on a television or a computer monitor can be used to activate the biophotonic compositions, wherein the display screen can emit light having an emission spectrum which overlaps an absorption spectrum of a photoactive agent in the photoactivatable compositions.

In some embodiments, the chromophore or combination of chromophores can be photoactivated by ambient light which may originate from the sun or other light sources. Ambient light can be considered to be a general illumination that comes from all directions in a room that has no visible source. In some embodiments, the chromophore or combination of chromophores can be photoactivated by light in the visible range of the electromagnetic spectrum. Exposure times to ambient light may be longer than that to direct light.

In some embodiments, different sources of light can be used to activate the biophotonic compositions, such as a combination of ambient light and direct LED light.

The duration of the exposure to actinic light required will be dependent on the surface of the treated area, the severity of the condition that is being treated, the power density, wavelength and bandwidth of the light source, the thickness of the biophotonic composition, and the treatment distance from the light source. The illumination of the treated area by fluorescence may take place within seconds or even fragment of seconds, but a prolonged exposure period is beneficial to exploit the synergistic effects of the absorbed, reflected and reemitted light on the composition of the present disclosure and its interaction with the tissue being treated. In some embodiments, the time of exposure to actinic light of the tissue or skin or wound on which the biophotonic composition has been applied is a period of from about 1 second to 30 minutes. In some embodiments, the time of exposure to actinic light of the tissue or skin or wound on which the biophotonic composition has been applied is a period of from about 1 minute to about 30 minutes. In some embodiments, the time of exposure to actinic light of the tissue or skin or wound on which the biophotonic composition has been applied is a period of from about 1 minute to about 5 minutes. In another embodiment, the time of exposure is from about 1 second to about 5 minutes. In some embodiments, the time of exposure to actinic light of the tissue or skin or wound on which the biophotonic composition has been applied is a period of from about 20 seconds to about 5 minutes, or from about 60 seconds to about 5 minutes. In another embodiment, the time of exposure to actinic light of the tissue on which the biophotonic composition has been applied is a period of less than about 5 minutes. In some embodiments, the time of exposure to actinic light of the tissue or skin or wound on which the biophotonic composition has been applied is a period of from about 1 second to about 5 minutes, or from 20 seconds to about 5 minutes, or from about 60 seconds to about 5 minutes per cm² of the area to be treated, so that the total time of exposure of a 10 cm² area would be from 10 minutes to 50 minutes.

In some embodiments, the biophotonic composition is illuminated for a period from about 1 second to about 30 minutes. In some embodiments, light is applied for a period of from about 1 minute to 3 minutes, from about 1 second to about 30 seconds, from about 1 second to about 60 seconds, from about 15 seconds to about 45 seconds, from about 30 seconds to about 60 seconds, from about 0.75 minute to about 1.5 minutes, from 1 minute to about 2 minutes, from about 1.5 minutes to about 2.5 minutes, from about 2 minutes to about 3 minutes, from about 2.5 minutes to about 3.5 minutes, from about 3 minutes to about 4 minutes, from about 3.5 to about 4.5 minutes, from about 4 minutes to about 5 minutes, from about 5 minutes to about 10 minutes, from about 10 minutes to about 15 minutes, from about 15 minutes to about 20 minutes, from about 20 to about 25 minutes, or from about 20 to about 30 minutes. In some embodiments, light is applied for a period of about 1 second. In some embodiments, light is applied for a period of about 5 seconds.

In some embodiments, light is applied for a period of about 10 seconds. In some embodiments, light is applied for a period of about 20 seconds. In some embodiments, light is applied for a period of about 30 seconds. In some embodiments, the biophotonic composition is illuminated for a period less than about 30 minutes. In some embodiments, the biophotonic composition is illuminated for a period less than about 20 minutes. In some embodiments, the biophotonic composition is illuminated for a period less than less than about 15 minutes. In some embodiments, the biophotonic composition is illuminated for a period less than less than about 10 minutes. In some embodiments, the biophotonic composition is illuminated for a period less than less than about 5 minutes. In some embodiments, the biophotonic composition is illuminated for a period less than less than about 1 minute.

In some embodiments, the biophotonic composition is illuminated for a period less than 30 seconds. In some embodiments, the biophotonic composition is illuminated for a period less than about 20 seconds. In some embodiments, the biophotonic composition is illuminated for a period less than about 10 seconds. In some embodiments, the biophotonic composition is illuminated for a period less than about 5 seconds. In some embodiments, the biophotonic composition is illuminated for a period less than about 1 second.

In some embodiments, the source of actinic light is in continuous motion over the treated area for the appropriate time of exposure. In some embodiments, multiple applications of the biophotonic composition and actinic light are performed. In some embodiments, the tissue, skin or wound is exposed to actinic light at least two, three, four, five or six times. In some embodiments, the tissue, skin, or wound is exposed to actinic light at least two, three, four, five, or six times with a resting period in between each exposure. In certain such embodiments, the resting period is less than about 1 minute, less than about 5 minutes, less than about 10 minutes, less than about 20 minutes, less than about 30 minutes, less than about 40 minutes, less than about 60 minutes, less than about 2 hours, less than about 4 hours, or less than about 6 hours. In some embodiments, the entire treatment may be repeated in its entirety as may be required by the patient. In some embodiments, a fresh application of the biophotonic composition is applied before another exposure to actinic light.

In the methods of the present disclosure, the biophotonic composition may be optionally removed from the site of treatment following application of light. In some embodiments, the biophotonic composition is left on the treatment site for more than about 30 minutes, more than about one hour, more than about 2 hours, or more than about 3 hours. It can be illuminated with ambient light. To prevent drying, the composition can be covered with a transparent or translucent cover such as a polymer film, or an opaque cover which can be removed before illumination.

For any of the methods described herein, the embodiments of this disclosure contemplate the use of any of the compositions, or mixtures of them, described throughout the application. In addition, in various embodiments of any of the methods described herein, combinations of any step or steps of one method with any step or steps from another method may be employed.

### Pyoderma, Deep Pyoderma, and Antibiotic-Resistant Pyoderma

The biophotonic compositions and methods of the present disclosure are useful to treat pyoderma, deep pyoderma, and antibiotic-resistant pyoderma. Therefore, it is an objective of the present disclosure to provide a method of providing biophotonic therapy to a target site, wherein the method is for the treatment of pyoderma, deep pyoderma, or antibiotic-resistant pyoderma.

Pyoderma is a bacterial infection of the skin that is very common in dogs and less common in cats. Several types of pyoderma exist:

Surface pyoderma, which is excessive bacterial proliferation confined to the skin surface (skin fold pyoderma and acute moist dermatitis);

Superficial pyoderma, where the bacterial infection is present in the hair follicles, without invasion of the dermis (bacterial folliculitis, mucocutaneous pyoderma and impetigo);

Deep pyoderma, in which the infectious process has gone beyond the basal membrane, and deeply involves the dermis, with the formation of piogranulomatous (boils) or diffuse (cellulite) lesions, that both tend to fistulize. The location of the lesions determines their classification. Classifications include: nasal furunculosis, chin furunculosis, interdigital or podal furunculosis, pyotraumatic furunculosis, furunculosis and cellulitis localized or generalized.

While surface and superficial pyoderma do not represent a serious problem for the veterinary dermatologist, as they are generally responsive to antibiotic therapy (topical and/or systemic), deep pyoderma is still a difficult problem that necessitates systemic antibiotic treatment lasting several weeks/months. Furthermore, another serious problem is antibiotic resistant pyoderma (so-called methicillin-resistant bacteria).

Symptoms of pyoderma include itchiness; pustules; crusted skin; small, raised lesions; loss of hair; and dried discharge in the affected area. Pyoderma occurs when the skin's surface has been broken, the skin has become injured due to chronic exposure to moisture, the normal skin bacteria have been altered or changed, the blood flow to the skin has become impaired or the immune system has been suppressed. Pyoderma is often secondary to allergic dermatitis and develops in the abrasions on the skin's surface that occur as a result of scratching. Puppies often develop “puppy pyoderma” in thinly haired areas such as the groin and underarms. Fleas, ticks, yeast or fungal skin infections, thyroid disease or hormonal imbalances, heredity and many medications may increase the risk of dogs and cats developing pyoderma. Additionally, dogs and cats with short coats, skin folds, and pressure calluses can be at a higher risk for pyoderma.

In some aspects, the disclosure provides a method of treating pyoderma, deep pyoderma, or antibiotic-resistant pyoderma comprising: applying a biophotonic composition to a patient in need thereof, wherein the biophotonic composition comprises at least one oxidant and at least one chromophore capable of activating the oxidant; and exposing said biophotonic composition to actinic light for a time sufficient for said chromophore to cause activation of said oxidant. In certain such aspects, the patient is a mammal, such as a feline or a canine.

In other aspects, the disclosure provides for use of a biophotonic composition for the manufacture of a medicament for treating a patient afflicted with pyoderma, deep pyoderma, or antibiotic-resistant pyoderma, wherein said composition comprises: at least one oxidant, and at least one chromophore capable of activating the oxidant; in association with a pharmacologically acceptable carrier. In certain such aspects, the patient is a mammal, such as a feline or a canine.

In some other aspects, the disclosure provides for use of a biophotonic composition for the treatment of a patient afflicted with pyoderma, deep pyoderma, or antibiotic resistant pyoderma, wherein said composition comprises: at least one oxidant; and at least one chromophore capable of activating the oxidant; in association with a pharmacologically acceptable carrier. In certain such aspects, the patient is a mammal, such as a feline or a canine.

The biophotonic compositions of the disclosure may be applied at regular intervals such as one or more times per week for one or more weeks, or at an interval deemed appropriate by the physician or veterinarian. In some embodiments, the biophotonic compositions of the disclosure are applied once per week for one or more weeks, such as once per week for one week, once per week for two weeks, once per week for three weeks, once per week for four weeks, once per week for five weeks, once per week for six weeks, once per week for seven weeks, or once per week for eight or more weeks.

In some embodiments, the biophotonic compositions of the disclosure are applied twice per week for one or more weeks, such as twice per week for one week, twice per week for two weeks, twice per week for three weeks, twice per week for four weeks, twice per week for five weeks, twice per week for six weeks, twice per week for seven weeks, twice per week for eight or more weeks.

In some embodiments, the biophotonic compositions of the disclosure are applied three times or more per week for one or more weeks, such as three times or more per week for one week, three or more times per week for two weeks, three times or more per week for three weeks, three times or more per week for four weeks, three times or more per week for five weeks, three times or more per week for six weeks, three times or more per week for seven weeks, three times or more per week for eight or more weeks.

In some embodiments, the biophotonic compositions and methods of the present disclosure are useful in treating pyoderma, for example, by ameliorating any symptom caused by a microorganism or inhibiting it from spreading. In some embodiments, the biophotonic compositions and methods of the present disclosure are useful in treating pyoderma, for example, by treating or preventing boils and lesions. In some embodiments, the biophotonic compositions and methods of the present disclosure are useful in treating pyoderma, for example, by treating or preventing antibiotic resistant bacteria. In some embodiments, the biophotonic compositions and methods of the present disclosure are useful in treating pyoderma without the use of antibiotics.

### Combination Therapies

Any of the biophotonic compositions, methods, or uses of this disclosure may be useful in combination with other therapeutics.

In some embodiments, the phrase “combination therapy” embraces the administration of the any of the compositions described herein, and an additional therapeutic agent, or mixtures of them, as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. The therapeutic agents can be administered by the same route or by different routes. For example, a first therapeutic agent of the combination selected may be administered by intravenous injection or orally while the biophotonic composition of the disclosure is administered topically. Alternatively, for example, all therapeutic agents may be administered topically. The sequence in which the therapeutic agents are administered is not narrowly critical. “Combination therapy” also can embrace the administration of the compositions as described herein in further combination with other biologically active ingredients (such as, but not limited to, a second and different therapeutic agent) and non-drug therapies (such as, but not limited to, surgery or radiation).

In some embodiments, the therapeutic agents administered in combination therapy simultaneously, separately, or sequentially with any of the compounds and compositions of this disclosure, or mixtures thereof, can comprise, but are not limited to: a non-steroidal anti-inflammatory drug (NSAID), an anti-inflammatory agent, a corticosteroid, an anti-allergic agent, a steroid drug, one or more of the antimicrobial agents described above, one or more collagens and/or agents that promote collagen synthesis described above, or mixtures thereof.

In some embodiments, any of the compositions described herein can allow the combination therapeutic agents and/or compositions described herein or mixtures thereof to be administered at a low dose, that is, at a dose lower than has been conventionally used in clinical situations.

Alternatively, the methods and combinations of this disclosure maximize the therapeutic effect at higher doses.

In some embodiments, when administered as a combination, the therapeutic agents can be formulated as separate compositions which are given at the same time or different times, or the therapeutic agents can be given as a single composition.

### Kits

The present disclosure also provides kits for preparing and/or applying any of the compositions of the present disclosure for the treatment of pyoderma, deep pyoderma, or antibiotic-resistant pyoderma. The kit may include a biophotonic topical composition, as defined above, and may also include a light source, an apparatus for applying or removing the composition, and instructions of use for the composition and/or a light source. In some embodiments, the biophotonic composition comprises at least one oxidant and at least one chromophore capable of activating the oxidant.

In some embodiments, the kit includes more than one composition, for example, a first and a second composition. The first composition may include at least one chromophore capable of activating the oxidant and the second composition may include at least one oxidant. In certain such embodiments, the oxidant is chosen from hydrogen peroxide, carbamide peroxide and benzoyl peroxide. In certain such embodiments, the first and/or second composition further comprises one or more gelling agents.

In some embodiments, the first composition may comprise the at least one chromophore capable of activating the oxidant in a liquid or as a powder, and the second composition may comprise at least one oxidant. In certain such embodiments, the oxidant is chosen from hydrogen peroxide, carbamide peroxide and benzoyl peroxide. In certain such embodiments, the first and/or second composition further comprises one or more gelling agents.

In some embodiments, the kit includes containers comprising the compositions of the present disclosure. In some embodiments, the kit includes a first container comprising the at least one chromophore capable of activating the oxidant, and a second container comprising at least one oxidant. In certain such embodiments, the oxidant is chosen from hydrogen peroxide, carbamide peroxide and benzoyl peroxide. In certain such embodiments, the first and/or second composition further comprises one or more gelling agents.

The containers may be light impermeable, air-tight and/or leak resistant. Exemplary containers include, but are not limited to, syringes, vials, or pouches. The first and second compositions may be included within the same container but separated from one another until a user mixes the compositions. In some embodiments, the container may be a dual-chamber syringe where the contents of the chambers mix on expulsion of the compositions from the chambers. In some embodiments, the pouch may include two chambers separated by a frangible membrane. In some embodiments, one component may be contained in a syringe and injectable into a container comprising the second component.

The biophotonic composition may also be provided in a container comprising one or more chambers for holding one or more components of the biophotonic composition, and an outlet in communication with the one or more chambers for discharging the biophotonic composition from the container.

In some embodiments, the kit comprises a systemic or topical drug for augmenting the treatment of the composition. For example, in certain such embodiments, the kit may include a systemic or topical antibiotic or hormone treatment for pyoderma, deep pyoderma, or antibiotic resistant pyoderma.

Written instructions on how to use the biophotonic composition in accordance with the present disclosure may be included in the kit, or may be included on or associated with the containers comprising the compositions of the present disclosure.

In some embodiments, the kit may comprise a further component which is a dressing. The dressing may be a porous or semi-porous structure for receiving the biophotonic composition. The dressing may comprise woven or non-woven fibrous materials.

In some embodiments of the kit, the kit may further comprise a light source such as a portable light with a wavelength appropriate to activate the chromophore in the biophotonic composition. The portable light may be battery operated or re-chargeable.

In some embodiments, the kit may further comprise one or more waveguides.

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present disclosure. They are not intended to limit or define the entire scope of this disclosure.

It should be appreciated that the disclosure is not limited to the particular embodiments described and illustrated herein but includes all modifications and variations falling within the scope of the disclosure as defined in the appended embodiments.

### General Protocol

Typically, the conventional treatment of deep pyoderma involves the administration of systemic antibiotics (injection or oral, often in combination), for variable periods of time as a function of the affected sites and severity of the lesions, which generally ranges from 4 to 9 weeks (28 days to 63 days). The typical treatment time for treating superficial pyoderma using the conventional therapy ranges from 4 to 6 weeks (28 days to 42 days). Studies were carried out on dogs with cutaneous infections to evaluate the efficacy of biophotonic therapy for treating pyoderma (e.g., deep or superficial pyoderma) in association with antibiotic therapy or as a mono-therapy. The studies were to determine whether the association of antibiotic therapy and biophotonic therapy is able to significantly reduce the period of conventional treatment of superficial and deep pyoderma.

A general protocol for applying the biophotonic therapy in the treatment of pyoderma in patients (i.e., dogs) comprises:

applying the composition of the present disclosure, wherein the composition comprises a carrier gel comprising 3%, 6%, or 12% urea peroxide (UP) (by weight of the total composition); a gelling agent; and water; and a chromophore gel comprising at least one chromophore (e.g., Eosin Y in an amount of about 109 μg/g of the total composition), a gelling agent, and water; illuminating the applied composition with an actinic light source, such as the THERA™ lamp, positioned at a distance from the treatment site of +/−5 cm for 2 minutes of illumination; and

optionally administering antibiotics to the same patient (e.g., dogs) being treated when necessary.

Patients, i.e., dogs, suffering from interdigital furunculosis or from furunculosis and cellulitis (localized or generalized) were recruited. For each category of disease, the patients were divided into five groups:

Group I was treated with systemic antibiotic therapy alone (cefadroxil 20 mg/kg PO BID, after verifying the effectiveness of cefadroxil on the basis of antibiotic susceptibility testing) for at least two weeks after full clinical resolution;

Group II was treated with both systemic antibiotic therapy (cefadroxil 20 mg/kg PO BID, after verifying the effectiveness of cefadroxil on the basis of antibiotic susceptibility testing) and biophotonic therapy once a week, for at least two weeks after full clinical resolution;

Group III was treated with both systemic antibiotic therapy (cefadroxil 20 mg/kg PO BID, after verifying the effectiveness of cefadroxil on the basis of antibiotic susceptibility testing) and biophotonic therapy twice a week, for at least two weeks after full clinical resolution.

Group IV was treated with biophotonic therapy (without antibiotics) once a week, for at least two weeks after full clinical resolution.

Group V was treated with biophotonic therapy (without antibiotics) twice a week, for at least two weeks after full clinical resolution.

In all patients, before treatment and every two weeks during treatment, skin swabs were performed and submitted for qualitative bacterial culture and to evaluate colony forming units (CFU). After clinical healing, all patients were monitored for 12 months to check for any relapse. Data were analyzed by the Student t-test for group comparisons of normally distributed variables with p<0.05 considered significant (Spatema A. 2008: “Dermatosi a carattere pustoloso e/o foruncoloso,” in: Spatema A. “Dermatologia del cane, dal segno clinic alla diagnosi e terapia,” Point Bet. Italie Ed., Milano, 139-170).

### Patients

Forty four (44) dogs were recruited and treated. Twenty five (25) dogs were affected by deep pyoderma: Among the twenty five (25) dogs, eight (8) dogs were treated with only antibiotics; five (5) dogs were treated with antibiotics and biophotonic therapy once a week; nine (9) dogs were treated with antibiotics and biophotonic therapy twice a week; three (3) dogs were treated with biophotonic therapy alone. Nineteen (19) dogs were affected by superficial pyoderma: seven (7) dogs were treated with antibiotics only; one (1) dog was treated with antibiotics and biophotonic therapy once a week; three (3) dogs were treated with antibiotics and biophotonic therapy twice a week; eight (8) dogs were treated with biophotonic therapy only.

### Results

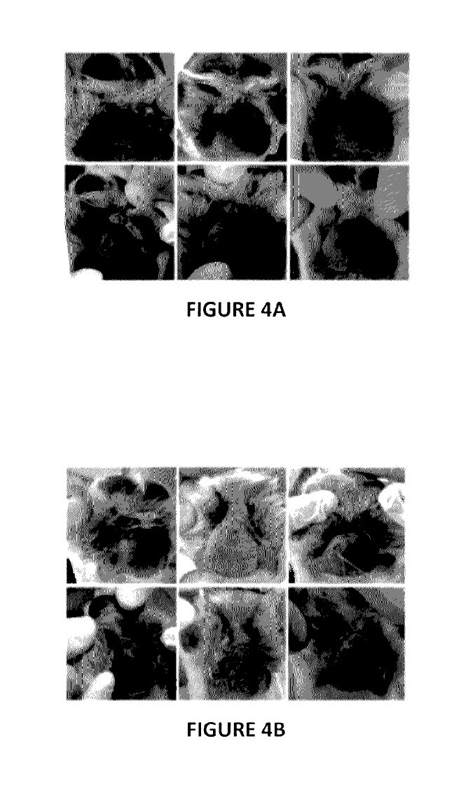

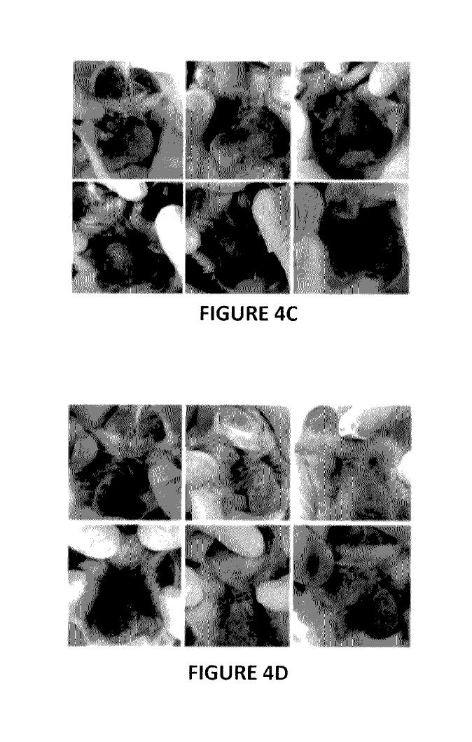

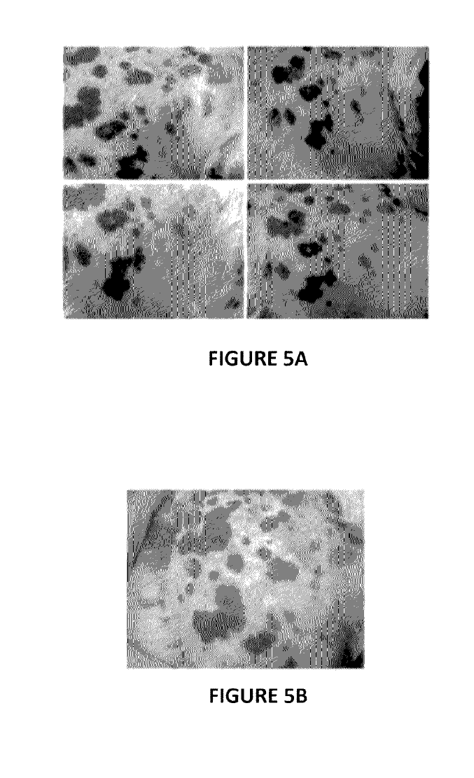

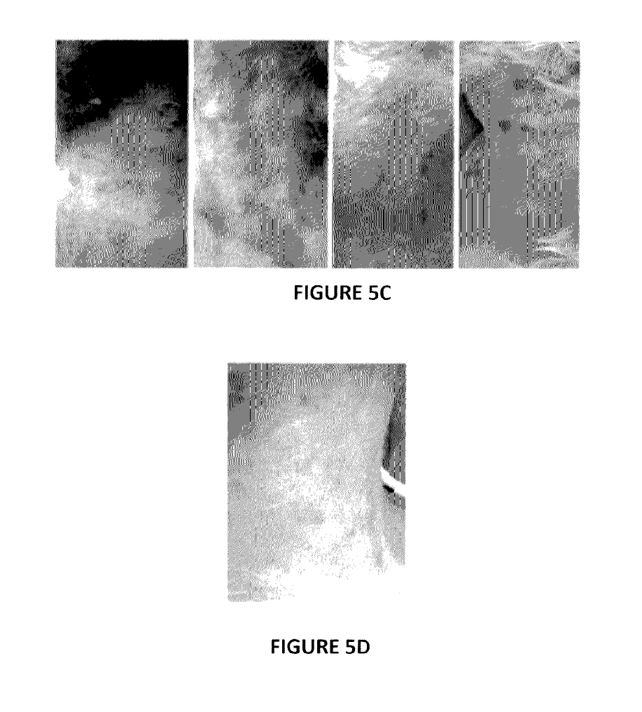

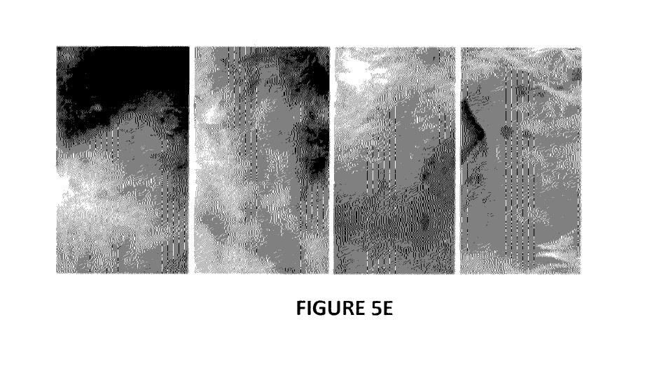

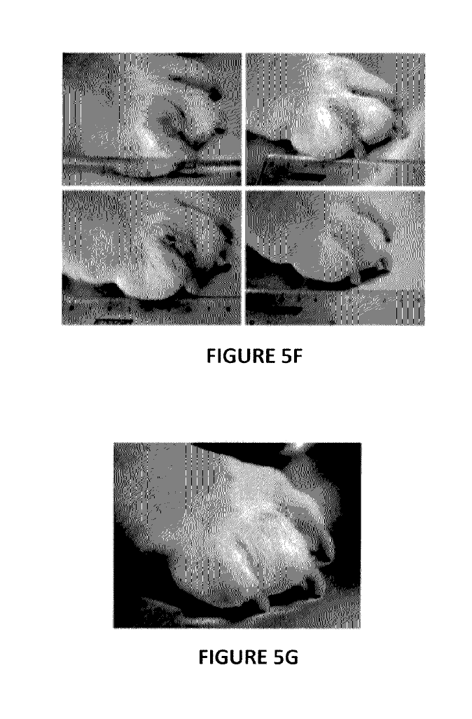

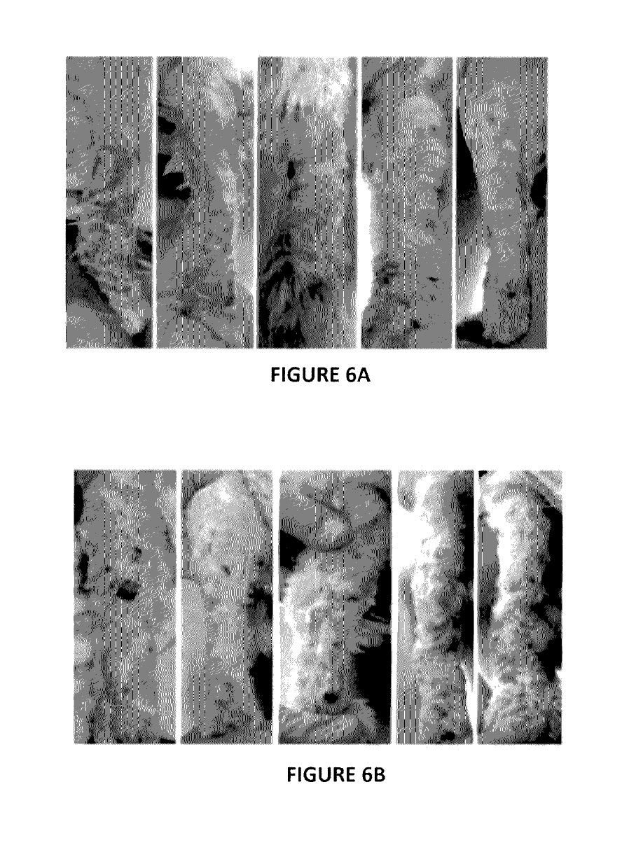

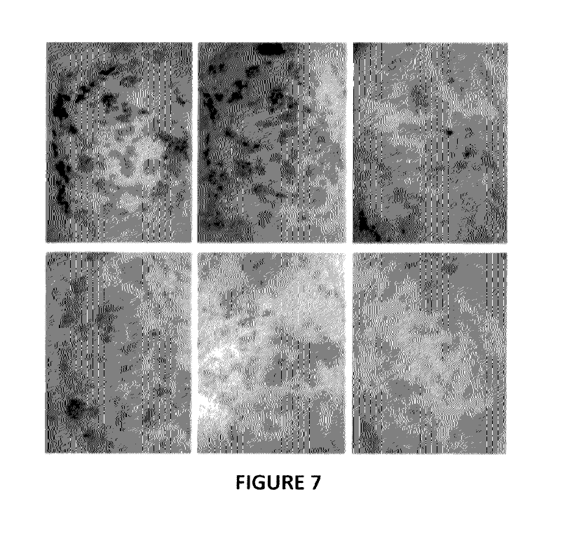

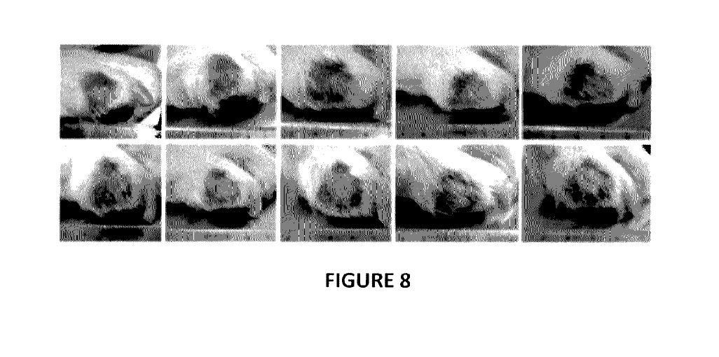

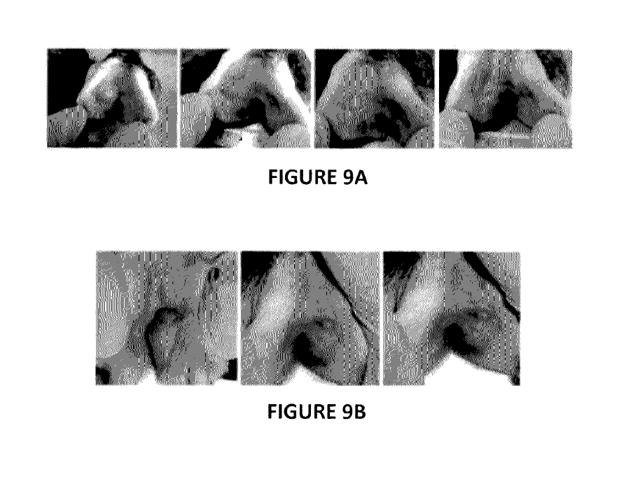

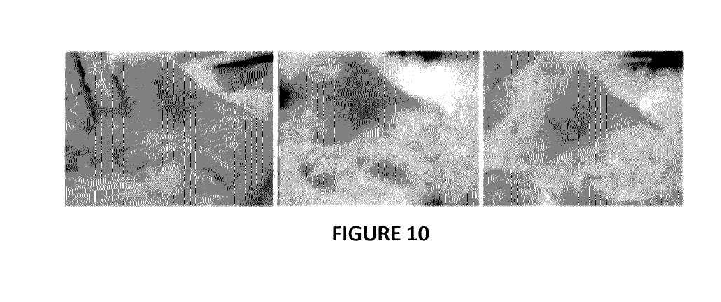

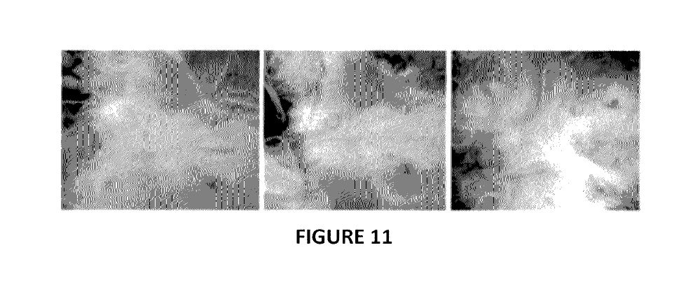

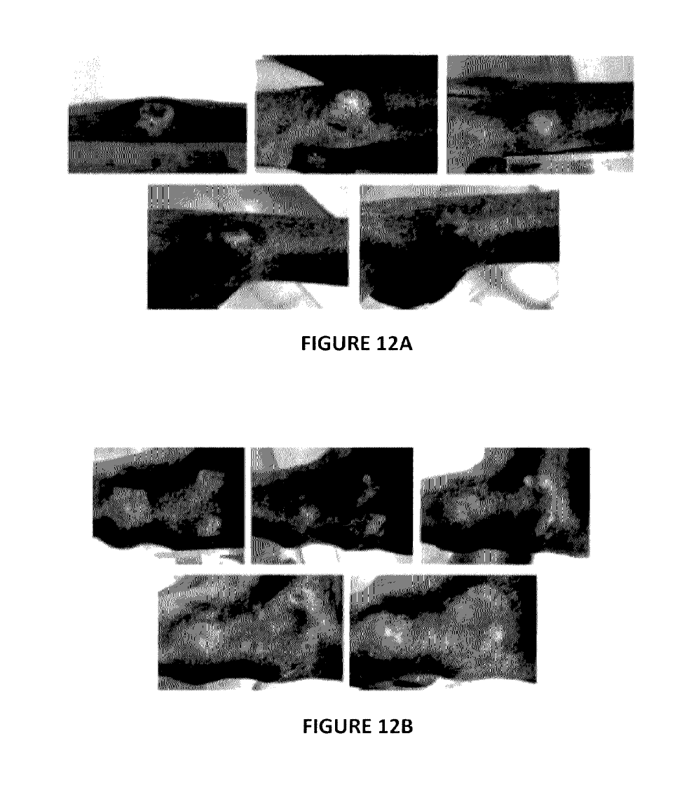

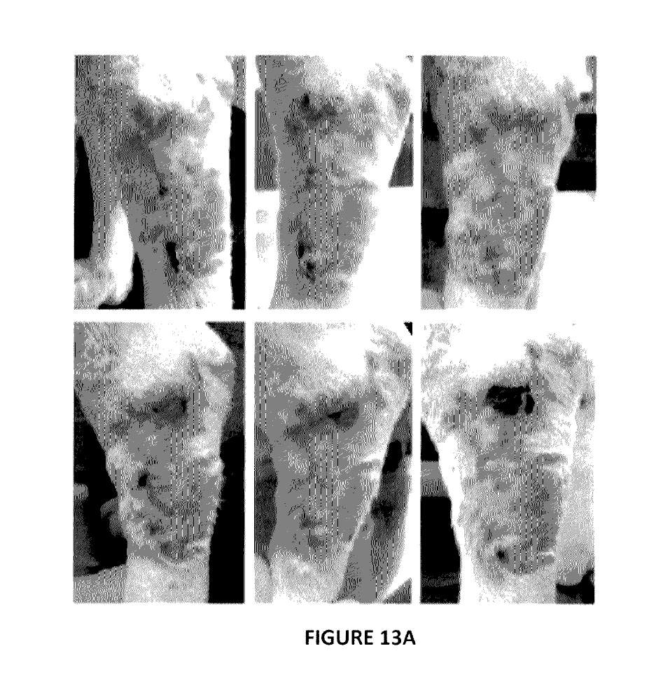

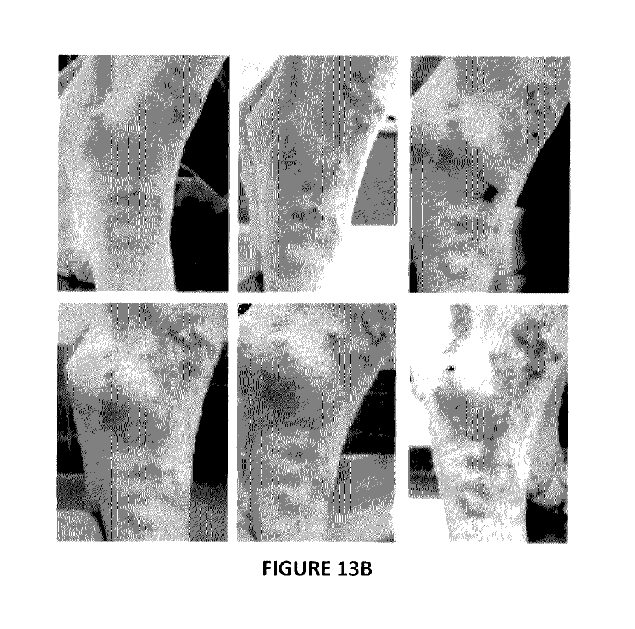

Example cases with treatment details were provided in the Table 1 below:

| | | | | Systemic | | |
|---|---|---|---|---|---|---|
| | | | | Antibiotic | | |
| | | | | Administered | | |
| | | | | (starting from the | | Visual |
| | | | | time of | | Observation |
| | | | Diseases/ | enrollment, | | with |
| | | | Pre- | cefadroxil, 20 mg/kg, | Treatment with | Progression |
| Case | | Age/ | treatment | twice a day | Biophotonic | of |
| No. | Breed | Year | Symptom | for three weeks) | Therapy | Treatment |
| 1 | Labrador | 7 | Recurrent | Yes | Previously | FIGS. 4A-4D |
| | retriever | | pyoderma | | treated with oral | |
| | | | due to food | | antibiotics and | |
| | | | allergy, with | | local therapy | |
| | | | interdigital | | with no results. | |
| | | | pustules, | | Left forelimb | |
| | | | lameness. | | foot was treated | |
| | | | Licking and | | with 12% UP and | |
| | | | scratching of | | illuminated with | |
| | | | the feet. | | actinic light; | |
| | | | | | Right forelimb | |
| | | | | | foot was treated | |
| | | | | | with 6% UP and | |
| | | | | | illuminated with | |
| | | | | | actinic light. | |
| | | | | | Left hindlimb | |
| | | | | | foot was treated | |
| | | | | | with 3% UP and | |
| | | | | | illuminated with | |
| | | | | | actinic light; | |
| | | | | | Right hindlimb | |
| | | | | | foot was | |
| | | | | | untreated as a | |
| | | | | | control. | |
| 2 | English | 6 | Superficial | Yes | All areas were | FIGS. 5A-5G |
| | Bulldog | | pyoderma in | | treated with 6% | |
| | | | dorsal and | | UP and | |
| | | | ventral | | chromophore gel; | |
| | | | region of the | | skins nearby to | |
| | | | neck and | | the areas to be | |
| | | | deep | | treated were | |
| | | | pyoderma at | | protected by four | |
| | | | the level of | | layers of surgical | |
| | | | right | | drape. | |
| | | | forelimb foot. | | Light source | |
| | | | No episodes | | applied was | |
| | | | of licking nor | | Thera ™ lamp at | |
| | | | scratching of | | a distance of 5 cm | |
| | | | the affected | | with an | |
| | | | areas | | exposition time | |
| | | | | | of 2 minutes and | |
| | | | | | the dog received | |
| | | | | | one treatment per | |
| | | | | | week. | |
| 3 | Shih-Tzu | 6 | Deep | Yes | All areas were | FIGS. 6A-6B |
| | | | pyoderma at | | treated with 6% | |
| | | | the level of | | UP and | |
| | | | the limbs. | | chromophore gel; | |
| | | | Licking and | | skin nearby to the | |
| | | | scratching of | | areas to be | |
| | | | the affected | | treated was | |
| | | | areas. | | protected by four | |
| | | | | | layers of surgical | |
| | | | | | drape. | |
| | | | | | Light source | |
| | | | | | applied was | |
| | | | | | Thera ™ lamp at | |
| | | | | | a distance of 5 cm | |
| | | | | | with an | |
| | | | | | exposition time | |
| | | | | | of 2 minutes and | |
| | | | | | the dog received | |
| | | | | | two treatments | |
| | | | | | per week | |
| 4 | Mixed | 10 | Furuncolosis | No | The area was | FIG. 7 |
| | breed dog | | at the level of | | treated with 6% | |
| | | | the dorsum. | | UP and | |
| | | | Licking and | | chromophore gel | |
| | | | scratching of | | for the first | |
| | | | the affected | | application but, | |
| | | | areas. | | soon after | |
| | | | | | treatment, skin | |
| | | | | | became inflamed | |
| | | | | | and the dog | |
| | | | | | scratched his | |
| | | | | | dorsum. From the | |
| | | | | | second treatment, | |
| | | | | | the area was | |
| | | | | | treated with 3% | |
| | | | | | UP. | |
| | | | | | Light source | |
| | | | | | applied was | |
| | | | | | Thera ™ lamp at | |
| | | | | | a distance of 5 cm | |
| | | | | | with an | |
| | | | | | exposition time | |
| | | | | | of 2 minutes and | |
| | | | | | the dog received | |
| | | | | | two treatments | |
| | | | | | per week. | |
| 5 | Labrador | 7 | Pyoderma of | No | The area was | FIG. 8 |
| | retriever | | right | | treated with 6% | |
| | dog | | hindlimb | | UP and | |
| | (same as | | foot. | | chromophore gel. | |
| | case 1, | | Licking of | | Light source | |
| | treating for | | the area. | | applied was | |
| | a different | | | | Thera ™ lamp at | |
| | foot) | | | | a distance of 5 cm | |
| | | | | | with an | |
| | | | | | exposition time | |
| | | | | | of 2 minutes and | |
| | | | | | the dog received | |
| | | | | | one treatment per | |
| | | | | | week. | |
| 6 | Boxer dog | 2 | Pyoderma of | Yes | The area was | FIGS. 9A-9B |
| | | | hindlimb | | treated with 6% | |
| | | | feet. | | UP and | |
| | | | | | chromophore gel. | |
| | | | | | Light source | |
| | | | | | applied was | |
| | | | | | Thera ™ lamp at | |
| | | | | | a distance of 5 cm | |
| | | | | | with an | |
| | | | | | exposition time | |
| | | | | | of 2 minutes and | |
| | | | | | the dog received | |
| | | | | | one treatment per | |
| | | | | | week. | |
| 7 | Bolognese | 8 | Superficial | No | The area was | FIG. 10 |
| | dog | | pyoderma of | | treated with 3% | |
| | | | right inguinal | | UP and | |
| | | | region. | | chromophore gel. | |
| | | | | | Light source | |
| | | | | | applied was | |
| | | | | | Thera ™ lamp at | |
| | | | | | a distance of 5 cm | |
| | | | | | with an | |
| | | | | | exposition time | |
| | | | | | of 2 minutes and | |
| | | | | | the dog received | |
| | | | | | one treatment per | |
| | | | | | week. | |
| 9 | Shih-Tzu | 4 | Furuncolosis | No | Inguinal region | FIG. 11 |
| | dog | | of inguinal | | was treated with | |
| | | | region. | | 6% UP and | |
| | | | Scratching of | | chromophore gel. | |
| | | | the affected | | Light source | |
| | | | areas | | applied was | |
| | | | | | Thera ™ lamp at | |
| | | | | | a distance of 5 cm | |
| | | | | | with an | |
| | | | | | exposition time | |
| | | | | | of 2 minutes and | |
| | | | | | the dog received | |
| | | | | | only one | |
| | | | | | treatment. | |
| 10 | Dobermann | 6 | Pyoderma of | No | Left hindlimb | FIGS. |
| | dog | | left hindlimb | | foot and tarsus | 12A-12B |
| | | | foot and | | were treated with | |
| | | | tarsus. | | 6% UP and | |
| | | | Licking or | | chromophore gel. | |
| | | | scratching of | | Light source | |
| | | | the affected | | applied was | |
| | | | areas. | | Thera ™ lamp at | |
| | | | | | a distance of 5 cm | |
| | | | | | with an | |
| | | | | | exposition time | |
| | | | | | of 2 minutes and | |
| | | | | | the dog received | |
| | | | | | one treatment per | |
| | | | | | week. | |
| 12 | Bull terrier | 4 | Deep | No | All areas were | FIGS. |
| | | | pyoderma in | | treated with 6% | 13A-13B |
| | | | right and left | | UP and | |
| | | | tarsus. | | chromophore gel. | |
| | | | No episodes | | Light source | |
| | | | of scratching | | applied was | |
| | | | of the | | Thera ™ lamp at | |
| | | | affected | | a distance of 5 cm | |
| | | | areas. | | with an | |
| | | | | | exposition time | |
| | | | | | of 2 minutes and | |
| | | | | | the dog received | |
| | | | | | two treatments | |
| | | | | | per week. | |

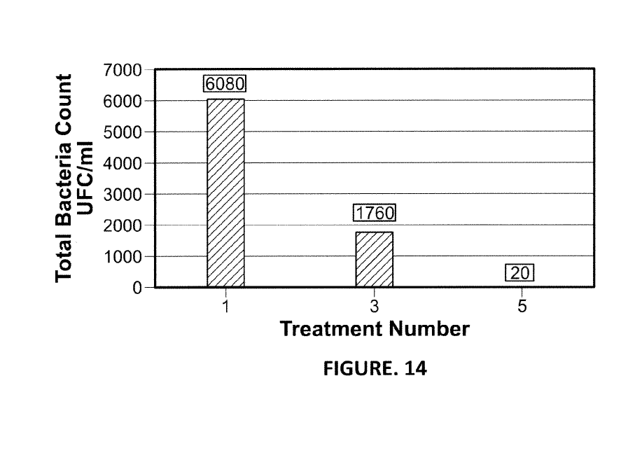

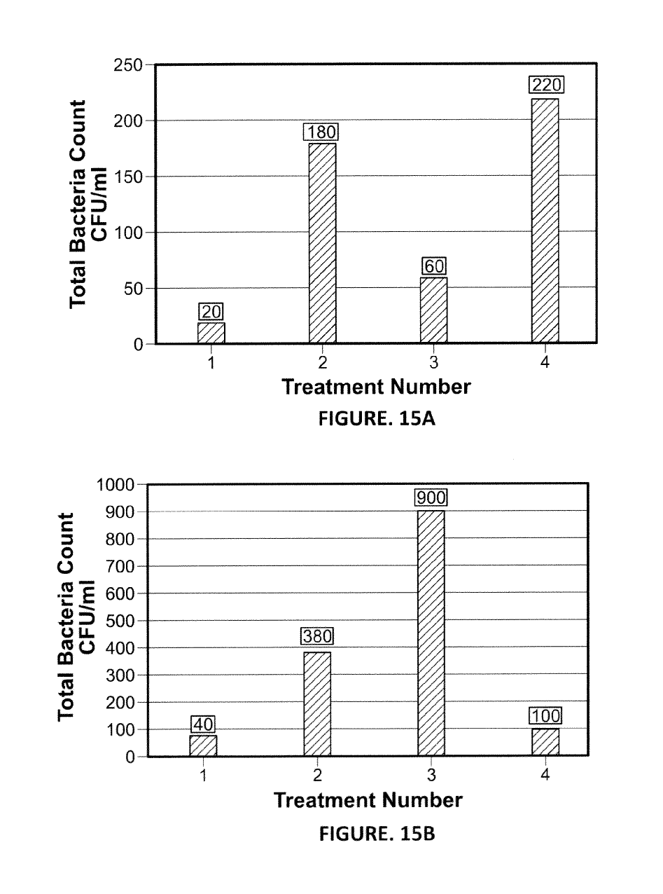

For cases 10 and 12, swabs for bacteriology were also performed. For case 10, isolated bacteria were Staphylococcus spp. beta hemolytic coagulase negative and Streptococcus spp. gamma hemolytic in the regions of lesion. For case 12, isolated bacteria included Staphylococcus spp. beta hemolytic coagulase negative; Enterococcus spp; and Proteus spp. in the regions of lesion. Total bacteria counts and their variations were shown in FIG. 14 for case 10 and FIGS. 15A and 15B for case 12.

The average length of biophotonic therapy treatment required for recovery are summarized as follows:

treating deep pyoderma in association with oral antibiotics required 5 to 6 weeks with 1 treatment per week;
treating deep pyoderma without administering antibiotics required 3 to 4 weeks with 2 treatments per week; and
treating superficial pyoderma without administering antibiotics required 1 to 3 weeks with 1 treatment per week.
Regarding the Average Length of Treatment for Deep or Superficial Pyoderma in Dogs with Biophotonic Therapy Alone or in Combination with Antibiotics

Dogs afflicted with deep pyoderma were treated with a biophotonic composition of this disclosure comprising 6% UP and chromophore gel, following the General Protocol as described above. Scoring of lesions was achieved considering the following parameters: papules, pustules, collarettes, crusts, alopecia and/or ulcers. Severity scale (0-4) was employed as follows: 1=mild, 2=moderate, 3=severe, 4=very severe.

TABLE 2
Length of treatment for treating deep pyoderma in dogs

| | Number of | Number of | Time of treatment | Severity |
|---|---|---|---|---|
| | treatments until | treatment per | until total | of the |
| Case # | resolution | week | resolution (weeks) | pyoderma |
| 2* | 4 | 1 | 4 | 3 |
| 3* | 5 | 1 | 5 | 4 |
| 5*** | 10 | 1 | 10 | 4 |
| 6* | 4 | 1 | 4 | 3 |
| 10*** | 5 | 1 | 5 | 3 |
| 12*** | 14 | 2 | 7 | 4 |
| 13** | 8 | 2 | 4 | 4 |
| 14** | 6 | 2 | 3 | 3 |
| 17** | 16 | 2 | 8 | 4 |
| 19** | 8 | 2 | 4 | 3 |
| 21** | 8 | 2 | 4 | 4 |
| 22* | 8 | 1 | 8 | 3 |
| 23** | 8 | 2 | 4 | 3 |
| Average | 8 | | 5.38 | |

*Antibiotic and biophotonic therapy once a week
**Antibiotics and biophotonic therapy twice a week
***Biophotonic therapy only

TABLE 2
Average length of treatment for treating deep pyoderma in dogs

| | | Mean | |
|---|---|---|---|
| | | Number of | Mean Number of |
| | Number of | Treatments | Weeks of Treating |
| | Treatment | until | until Total |
| Treatment types | per Week | Resolution | Resolution |
| Biophotonic therapy and | Once | 5.2 | 5.2 |
| antibiotics | | | |
| Biophotonic therapy only | Once | 8.0 | 8.0 |
| Biophotonic therapy and | Twice | 7.5 | 3.75 |
| antibiotics | | | |
| Biophotonic therapy only | Twice | >11 | >5.5 |

Similarly, Dogs afflicted with superficial pyoderma were treated with a biophotonic composition of this disclosure containing 6% UP and chromophore gel, following the General Protocol as described above, without administration of oral antibiotics, except for case 16.

TABLE 3
Length of treatment for treating superficial pyoderma in dogs

| | Number of | Number of | Time of treatment | Severity |
|---|---|---|---|---|
| | treatments | treatment | until total resolution | of the |
| Case # | until resolution | per week | (weeks) | pyoderma |
| 7^{Ψ} | 3 | 1 | 3 | 4 |
| 8^{Ψ} | 2 | 1 | 2 | 5 |
| 9^{Ψ} | 1 | 1 | 1 | 5 |
| 11^{Ψ} | 2 | 1 | 2 | 4 |
| 15** | 6 | 2 | 3 | 5 |
| 16*** | 4 | 2 | 2 | 4 |
| 18^{±} | 3 | 2 | 1.5 | 3 |
| 20*** | 4 | 2 | 2 | 5 |
| 40 | 4 | 1 | 4 | 5 |
| Average | 3.22 | | 2.28 | |

**Antibiotics and biophotonic therapy twice a week
***Biophotonic therapy only
^{Ψ}Biophotonic therapy once a week
^{±}Biophotonic therapy twice a week

TABLE 4
Average length of treatment for treating superficial pyoderma in dogs

| | | Mean | |
|---|---|---|---|
| | Number of | Number of | Mean Number of |
| | Treatment | Treatments | Weeks of Treating |
| | per | until | until Total |
| Treatment types | Week | Resolution | Resolution |
| Biophotonic therapy and | Once | 4.0 | 4.0 |
| antibiotics | | | |
| Biophotonic therapy only | Once | 2.4 | 2.4 |
| Biophotonic therapy and | Twice | 4.0 | 2.0 |
| antibiotics | | | |
| Biophotonic therapy only | Twice | 4.25 | 2.13 |

In summary, biophotonic therapy has been proven effective in all cases, proving its reliability and effectiveness in treating both deep and superficial pyoderma in dogs. For instance, dogs which were affected by deep pyoderma and treated with both biophotonic therapy and oral antibiotic achieved healing in less than 5.5 weeks (instead of 4-9 weeks as required by conventional therapy), without significant differences with respect to the administration of the treatment once or twice per week. Dogs which were affected by superficial pyoderma and treated only with biophotonic therapy achieved healing in less than 2.5 weeks (instead of 4-6 weeks as required by conventional therapy) and the administration of the treatment twice per week has proved to be even more effective. No recurrence in the same areas was recorded in patients treated with biophotonic compositions of this disclosure.

While embodiments of the disclosure have been described above and illustrated in the accompanying figures, it will be evident to those skilled in the art that modifications may be made therein without departing from the essence of this disclosure. Such modifications are considered as possible variants comprised in the scope of the disclosure.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A method of treating pyoderma, deep pyoderma, or antibiotic-resistant pyoderma comprising:
a) applying a biophotonic composition to a patient in need thereof, wherein the biophotonic composition comprises at least one oxidant and at least one chromophore capable of activating the oxidant; and
b) exposing said biophotonic composition to actinic light for a time sufficient for said chromophore to cause activation of said oxidant.

2. The method according to claim 1, wherein the patient is a mammal.

3. The method according to claim 2, wherein the mammal is a canine.

4. The method according to claim 2, wherein the mammal is a feline.

5. The method according to any one of claims 1 to 4, wherein the composition is applied to the patient's skin.

6. The method according to any one of claims 1 to 5, wherein said biophotonic composition is exposed to actinic light for a period of less than about 5 minutes.

7. The method according to any one of claims 1 to 5, wherein said biophotonic composition is exposed to actinic light for a period of from about 1 second to about 5 minutes.

8. The method according to any one of claims 1 to 7, wherein said biophotonic composition is exposed to actinic light for a period of less than about 5 minutes per cm² of an area to be treated.

9. The method according to any one of claims 1 to 7, wherein said biophotonic composition is exposed to actinic light for a period of about 1 second to about 5 minutes per cm² of an area to be treated.

10. The method according to any one of claims 1 to 9, wherein the source of actinic light is positioned over an area to be treated.

11. The method according to any one of claims 1 to 10, wherein said actinic light is visible light having a wavelength between about 400 nm and about 700 nm.

12. The method according to any one of claims 1 to 11, wherein the oxidant is chosen from hydrogen peroxide, carbamide peroxide and benzoyl peroxide.

13. The method according to any one of claims 1 to 11, wherein the oxidant is chosen from a peroxy acid and an alkali metal percarbonate.

14. The method according to any one of claims 1 to 13, wherein the composition further comprises at least one healing factor chosen from hyaluronic acid, glucosamine and allantoin.

15. The method according to any one of claims 1 to 14, wherein the composition further comprises at least one gelling agent.

16. The method according to claim 15, wherein the gelling agent is chosen from glucose, modified starch, methyl cellulose, carboxymethyl cellulose, propyl cellulose, hydroxypropyl cellulose, a carbomer, alginic acid, sodium alginate, potassium alginate, ammonium alginate, calcium alginate, agar, carrageenan, locust bean gum, pectin, and gelatin.

17. The method according to any one of claims 1 to 16, wherein the chromophore is chosen from a xanthene derivative dye, an azo dye, a biological stain, and a carotenoid.

18. The method according to claim 17, wherein said xanthene derivative dye is chosen from a fluorene dye, a fluorone dye, and a rhodole dye.

19. The method according to claim 18, wherein said fluorene dye is chosen from a pyronine dye and a rhodamine dye.

20. The method according to claim 19, wherein said pyronine dye is chosen from pyronine Y and pyronine B.

21. The method according to claim 19, wherein said rhodamine dye is chosen from rhodamine B, rhodamine G and rhodamine WT.

22. The method according to claim 18, wherein said fluorone dye is chosen from fluorescein and fluorescein derivatives.

23. The method according to claim 22, wherein said fluorescein derivative is chosen from phloxine B, rose bengal, and merbromine.

24. The method according to claim 22, wherein said fluorescein derivative is chosen from Eosin Y, Eosin B and Erythrosine B.

25. The method according to claim 24, wherein said fluorescein derivative is Eosin Y.

26. The method according to claim 17, wherein said azo dye is chosen from methyl violet, neutral red, para red, amaranth, carmoisine, allura red AC, tartrazine, orange G, ponceau 4R, methyl red, and murexide-ammonium purpurate.

27. The method according to claim 17, wherein said biological stain is chosen from saffranin O, basic fuchsin, acid fuschin, 3,3′ dihexylocarbocyanine iodide, carminic acid, and indocyanine green.

28. The method according to claim 17, wherein said carotenoid is chosen from crocetin, a-crocin (S,S-diapo-S,S-carotenoic acid), zeaxanthin, lycopene, α-carotene, β-carotene, bixin, and fucoxanthine.

29. The method according to claim 17, wherein said carotenoid is present in the composition as a mixture chosen from saffron red powder, annatto extract and brown algae extract.

30. The method according to any one of claims 1 to 29, wherein said composition further comprises at least one chelating agent chosen from ethylenediaminetetraacetic acid (EDTA) and ethylene glycol tetraacetic acid (EGTA).

31. The method according to any one of claims 1 to 30, wherein steps a) and b) are performed once per week for one week.

32. The method according to any one of claims 1 to 30, wherein steps a) and b) are performed once per week for two weeks.

33. The method according to any one of claims 1 to 30, wherein steps a) and b) are performed once per week for three weeks.

34. The method according to any one of claims 1 to 30, wherein steps a) and b) are performed once per week for four weeks.

35. The method according to any one of claims 1 to 30, wherein steps a) and b) are performed once per week for five weeks.

36. The method according to any one of claims 1 to 30, wherein steps a) and b) are performed once per week for six weeks.

37. The method according to any one of claims 1 to 30, wherein steps a) and b) are performed twice per week for one week.

38. The method according to any one of claims 1 to 30, wherein steps a) and b) are performed twice per week for two weeks.

39. The method according to any one of claims 1 to 30, wherein steps a) and b) are performed twice per week for three weeks.

40. The method according to any one of claims 1 to 30, wherein steps a) and b) are performed twice per week for four weeks.

41. The method according to any one of claims 1 to 30, wherein steps a) and b) are performed twice per week for five weeks.

42. The method according to any one of claims 1 to 30, wherein steps a) and b) are performed twice per week for six weeks.

43. Use of a biophotonic composition for the manufacture of a medicament for treating a patient afflicted with pyoderma, deep pyoderma, or antibiotic-resistant pyoderma, wherein said composition comprises:
at least one oxidant, and
at least one chromophore capable of activating the oxidant;
in association with a pharmacologically acceptable carrier.

44. Use of a biophotonic composition for the treatment of a patient afflicted with pyoderma, deep pyoderma, or antibiotic-resistant pyoderma, wherein said composition comprises:
at least one oxidant; and
at least one chromophore capable of activating the oxidant;
in association with a pharmacologically acceptable carrier.

45. The use according to claim 43 or 44, wherein the patient is a mammal.

46. The use according to claim 45, wherein the mammal is a canine.

47. The use according to claim 46, wherein the mammal is a feline.

48. The use according to any one of claims 43 to 47, wherein the composition further comprises at least one healing factor chosen from hyaluronic acid, glucosamine and allantoin.

49. The use according to any one of claims 43 to 48, wherein the oxidant is chosen from hydrogen peroxide, carbamide peroxide and benzoyl peroxide.

50. The use according to any one of claims 43 to 48, wherein the oxidant is chosen from a peroxy acid and an alkali metal percarbonate.

51. The use according to any one of claims 43 to 50, wherein the composition further comprises at least one gelling agent.

52. The use according to claim 51, wherein the gelling agent is chosen from glucose, modified starch, methyl cellulose, carboxymethyl cellulose, propyl cellulose, hydroxypropyl cellulose, a carbomer, alginic acid, sodium alginate, potassium alginate, ammonium alginate, calcium alginate, agar, carrageenan, locust bean gum, pectin, and gelatin.

53. The use according to any one of claims 43 to 52, wherein the chromophore is chosen from a xanthene derivative dye, an azo dye, a biological stain, and a carotenoid.

54. The use according to claim 53, wherein said xanthene derivative dye is chosen from a fluorene dye, a fluorone dye, and a rhodole dye.

55. The use according to claim 54, wherein said fluorene dye is chosen from a pyronine dye and a rhodamine dye.

56. The use according to claim 55, wherein said pyronine dye is chosen from pyronine Y and pyronine B.

57. The use according to claim 55, wherein said rhodamine dye is chosen from rhodamine B, rhodamine G and rhodamine WT.

58. The use according to claim 54, wherein said fluorone dye is chosen from fluorescein and fluorescein derivatives.

59. The use according to claim 58, wherein said fluorescein derivative is chosen from phloxine B, rose bengal, and merbromine.

60. The use according to claim 58, wherein said fluorescein derivative is chosen from eosin Y, eosin B and erythrosine B.

61. The use according to claim 60, wherein said fluorescein derivative is Eosin Y.

62. The use according to claim 53, wherein said azo dye is chosen from methyl violet, neutral red, para red, amaranth, carmoisine, allura red AC, tartrazine, orange G, ponceau 4R, methyl red, and murexide-ammonium purpurate.

63. The use according to claim 53, wherein said biological stain is chosen from saffranin O, basic fuchsin, acid fuschin, 3,3′ dihexylocarbocyanine iodide, carminic acid, and indocyanine green.

64. The use according to claim 53, wherein said carotenoid is chosen from crocetin, a-crocin (S,S-diapo-S,S-carotenoic acid), zeaxanthin, lycopene, α-carotene, β-carotene, bixin, and fucoxanthine.

65. The use according to claim 53, wherein said carotenoid is present in the composition as a mixture chosen from saffron red powder, annatto extract and brown algae extract.

66. The use according to any one of claims 43 to 65, wherein said composition further comprises at least one chelating agent chosen from ethylenediaminetetraacetic acid (EDTA) and ethylene glycol tetraacetic acid (EGTA).

67. The use according to any of claims 43 to 66, wherein the patient is treated once per week for one or more weeks.

68. The use according to any one of claims 43 to 66, wherein the patient is treated twice per week for one or more weeks.
